(19) United States
(12) Patent Application Publication
(10) Pub. No.: US 2022/0033450 A1
(43) Pub. Date: Feb. 3, 2022

(54) VIRALLY EXPRESSED INHIBITORS OF PDZ DOMAINS, SUCH AS PICK1 AND USES THEREOF

(71) Applicant: University of Copenhagen, Copenhagen K (DK)

(72) Inventors: Andreas Toft Sørensen, Gentofte (DK); Kenneth L. Madsen, Virum (DK); Nikolaj Riis Christensen, Allerød (DK); Kristian Strømgaard, Roskilde (DK)

(73) Assignee: University of Copenhagen, Copenhagen K (DK)

(21) Appl. No.: 17/283,995

(22) PCT Filed: Oct. 22, 2019
(86) PCT No.: PCT/EP2019/078736

§ 371 Date: Apr. 9, 2021

(30) Foreign Application Priority Data
Oct. 22, 2018 (EP) 18201742.6

(51) Int. Cl. C07K 14/47; C07K 7/06; C12N 15/86; A61P 25/00; A61P 25/04; A61P 25/28

(52) U.S. Cl. CPC C07K 14/47; C07K 7/06; C12N 15/86; A61P 25/00; A61K 38/00; A61P 25/28; C07K 2319/73; C12N 2750/14143; A61P 25/04

(57) ABSTRACT

The present invention relates to virally expressed peptides with high affinity for the PDZ domains, such as the PDZ domain of PICK1. The invention furthermore relates to the therapeutic use of these peptides in prevention and/or treatment of diseases and/or disorders associated with maladaptive plasticity and/or transmission.

### CROSS REFERENCE TO RELATED APPLICATIONS

This application is a U.S. National Stage under 35 U.S.C. § 371 of international application PCT/EP2019/078736 filed Oct. 22, 2019, which claims priority to European Application No: 18201742.6 filed Oct. 22, 2018, the entire contents of each of which are incorporated herein by reference.

### SEQUENCE LISTING

This application incorporates by reference a Sequence Listing as an ASCII text file entitled “2021-08-19 Corrected Sequence listing 16COPE-HO78102PA.txt” created on Aug. 19, 2021 having a size of 19,123 bytes.

### TECHNICAL FIELD

The present invention relates to virally expressed peptides which bind to PDZ domains, such as the Protein Interacting with C Kinase-1 (PICK1) and thereby block PDZ domains mediated, e.g. PICK1-mediated, protein-protein interactions. The invention furthermore relates to therapeutic use of said peptides.

### BACKGROUND

Protein-protein interactions (PPIs) are vital for most biochemical and cellular processes and are often mediated by scaffold and signal transduction complexes. One of the most abundant classes of human facilitators of PPIs is the family of postsynaptic density protein-95 (PSD-95)/Discs-large/ZO-1 (PDZ) domains. Protein Interacting with C Kinase-1 (PICK1) is an intracellular scaffold protein primarily involved in regulation of protein trafficking and cell migration by mediating and facilitating PPIs via its two PDZ domains. Central to PICK1's cellular role is its ability to bind and interact with numerous intracellular molecules including various protein partners, as well as membrane phospholipids. PDZ domain proteins in the postsynaptic density, which dynamically regulate the surface expression and activity of the glutamate receptors, represent attractive alternative drug targets, but it has proven challenging to develop sufficiently potent small molecule inhibitors and peptide drugs generally suffer from poor pharmacokinetic profiles. PICK1 is another PDZ domain containing scaffolding protein that plays a central role in synaptic plasticity. PICK1 is a functional dimer, with two PDZ domains flanking the central membrane binding BAR domain, which also mediates the dimerization. For example, the PICK1 PDZ domain interacts directly with the C-terminus of the GluA2 subunit of the AMPA receptors (AMPAR) as well as protein kinase A and C, thereby regulating AMPAR phosphorylation and surface expression and in turn synaptic plasticity tuning the efficacy of individual synapses. Synaptic plasticity serves as the molecular substrate for learning and memory. In diseased states, such as ischemia after stroke, neuropathic pain and addiction, abnormal synaptic stimulation and transmission cause maladaptive plasticity leading to hyper-sensitization of glutamatergic synapses in part through expression of calcium permeable (CP) AMPA-type glutamate receptors (CP-AMPARs).

Although numerous diseased states, including ischemia after stroke and head injury, amyotrophic lateral sclerosis (ALS), epilepsy, Alzheimer's disease, neuropathic pain, hearing disorders (e.g. tinnitus) and addiction, involve an over-activation or sensitization of the glutamate system, the NMDA receptor antagonists such as ketamine (anaesthetic) are, due to general problems with severe side effects, currently the only drugs in clinical use that target the glutamate system. There is thus a need for a treatment for diseases such as neuropathic pain, excitotoxicity following ischemia and drug addiction, three conditions that are currently without any effective therapy.

### SUMMARY

The present invention provides the design of a genetically encoded high affinity peptide inhibitor towards scaffolding proteins, such as for example protein interacting C kinase 1 (PICK1) or postsynaptic density-95 (PSD-95), for viral delivery, using for example adeno associated virus as a vehicle for genetic delivery. This invention differs from current glutamate receptor drugs by targeting the scaffolding proteins responsible for the trafficking of the receptor, rather than targeting the receptor directly. It differs from existing compounds targeting PDZ domains, such as PICK1, in that it can be delivered with high efficacy and selectivity as a single viral injection thus lifting therapeutic outcome, patient compliance in patients with conditions such as neuropathic pain, excitotoxicity following ischemia or drug addiction while reducing possible side effects.

In a first aspect, the present disclosure provides a polynucleotide comprising a sequence encoding upon expression a polypeptide comprising:

a) a first polypeptide part comprising or consisting of an amino acid sequence capable of forming a dimer; and
b) a second polypeptide part comprising or consisting of an amino acid sequence selected from the group consisting of Class I PDZ domains binding motifs (PBM), Class II PBM and Class III PBM,
wherein the first and the second polypeptides are optionally operably linked via a linker.

In one embodiment, the present invention provides a polynucleotide comprising a sequence encoding upon expression:

a) a first polypeptide comprising or consisting of an amino acid sequence capable of forming a leucine zipper; and b) a second polypeptide comprising or consisting of an amino acid sequence of the general formula (II):

(SEQ ID NO: 11)
Z₁Z₂Z₃Z₄Z₅;

wherein:
Z₁ is a proteogenic or non-proteogenic amino acid, preferably H, L, V, I, A; or is absent;
Z₂ is a proteogenic or non-proteogenic amino acid, preferably W, F, S, T; or is
absent;
Z₃ is a proteogenic or non-proteogenic amino acid, preferably L, V, I, F; A, Y;
Z₄ is a proteogenic or non-proteogenic amino acid, preferably K, S, T, R; and
Z₅ is V, I, L or C.

In another aspect, the present invention provides a recombinant expression vector comprising the polynucleotide sequence according to the above aspect.

In another aspect, the present invention provides a polypeptide encoded by the polynucleotide, or the expression vector according to the above aspects.

In another aspect, the present invention provides a recombinant host cell comprising the polynucleotide or the expression vector according to the above aspects.

In another aspect, the present invention provides a composition comprising the polynucleotide, the expression vector or polypeptide according the above aspects.

In another aspect, the present invention provides the polynucleotide, the expression vector, the polypeptide, the cell, and/or the composition according to the above aspects, for use as a medicament.

In another aspect, the present invention provides the polynucleotide, the expression vector, the polypeptide, the cell, and/or the composition according to the above aspects, for use in treatment of a disease and/or disorder associated with maladaptive plasticity and/or transmission.

In another aspect, the present invention provides a method of treatment or prevention of a disease and/or disorder associated with maladaptive plasticity and/or transmission in a subject in need thereof, comprising administering a therapeutically effective amount of the polynucleotide, the expression vector, the polypeptide, the cell, and/or the composition according to the above aspects.

In another aspect, the present invention provides a use of the polynucleotide, the expression vector, the polypeptide, the cell, and/or the composition according to any one of above aspects, for the manufacture of a medicament for the treatment of a disease and/or disorder associated with maladaptive plasticity and/or transmission.

### DESCRIPTION OF DRAWINGS

FIG. 1: Development of a high affinity, genetically encoded inhibitor of PICK1 The Primary sequence of GCN4-GS4-C5 and variants were used. The sequence comprises an N-terminal part GCN4 (GCN4p1) from a yeast bZIP transcription factor mediating potent dimerization (as shown), a GS linker sequence to position the C5 regions from the human dopamine transporter (HWLKV), which is one sequence that retain favorable PICK1 PDZ domain affinity. Variants of the primary structure with Proline (P) in position 7 and 14, and with a C-terminal Aspartate (D) (to compromise PICK1 PDZ domain binding) are illustrated.

FIG. 2: Dimeric assembly of GCN4-GS4-C5 Size exclusion chromatography of synthetic GCN4-GS4-C5 demonstrated elution profile at ˜18.3 ml corresponding to a dimeric assembly of the peptide, whereas GCN4-GS4-7P14P was partly shifted to elution at 19.7 ml corresponding to a monomer. GCN4-GS4-V-to-D eluted as a homodimer. The tested peptides contained an N-terminal Biotin and an Ahx (6-aminohexanoic acid) linker.

FIG. 3: GCN4-GS4-C5 induces a tetrameric configuration of PICK1 Size exclusion chromatography of (A) purified PICK1 (40 μM) in absence of peptide eluting at 11.7 ml that corresponds to the dimeric protein. (B) PICK1 (40 μM) incubated with GCN4-GS4-C5 (10 μM) showed induction of tetramer configuration of PICK1 eluting at 10 ml. (C) PICK1 incubated with GCN4-GS4-V-to-D (10 μM), eluted exclusively in the dimeric peak of PICK1 and the profile further showed unbound peptide at ˜18 ml. (D) PICK1 incubated with GCN4-GS4-7P14P eluted in the dimeric peak of PICK1 and showed no unbound peptide. Absorbance values have been normalized for clarity. The tested peptides contained an N-terminal Biotin and an Ahx linker.

FIG. 4: GCN4-GS4-C5 shows high affinity for PICK1 Fluorescence polarization binding of GCN4 peptides to PICK1 using 5FAM DATCS as tracer (20 nM). Binding curve of GCN4-GS4-C5 gave an apparent affinity of 152 nM corresponding to a 32-fold increase compared to the monomeric DAT C5 peptide. The binding curve for GCN4-GS4-7P14P was shifted to higher concentration, indicating a lower affinity (˜5-fold) compared to GCN4-GS4-C5. GCN4-GS4-V-to-D did not show any binding up to 100 μM. The tested peptides contained an N-terminal Biotin and an Ahx linker.

FIG. 5: GCN4-GS4-C5 interaction with PICK1 relies on the PDZ domain. Fluorescence polarization binding of GCN4-GS4-C5 to PICK1 Wt and A87L using PEG4 linked dimeric C5 as tracer (20 nM). PICK1 A87L, which has a mutation in the PDZ binding groove, demonstrated 18-fold reduced binding affinity compared to PICK1 wt. The tested GCN4-GS4-C5 peptide contained an N-terminal Biotin and an Ahx linker.

FIG. 6: A model of the self-assemble propensity of the dimeric structure of GCN4-GS4-C5 oligopeptide. A HA-tag was attached to the N-terminus of each GCN4-GS4-C5 oligopeptide.

FIG. 7: Intrathecal AAV8-hSyn-HA-GCN4-GS4-C5-WPRE-pA administered confers expression towards the grey matter of the spinal cord as revealed by positive HA-stain (seen as white area) at the spinal vertebra L1-L2 level (A; 10× objective). This expression appeared to be confined towards neurons within the spinal horn (B; marked by white arrows, 20× objective), as also predicted by the use of the pan-neuronal human Synapsin (hSyn) promoter.

FIG. 8: Time line for the behavioral experiment testing intrathecal AAV8-hSyn-HA-GCN4-GS4-C5-WPRE-pA administered in the spared nerve injury (SNI) model of neuropathic pain. Virus ith. inj. stands for virus intrathecal injection, whereas D stands for days.

FIG. 9: AAV8-hSyn-HA-GCN4-GS4-C5-WPRE-pA treatment completely and persistently alleviates mechanical hyperalgesia in the mouse SNI model Animals were injected intrathecally with saline, AAV8-hSyn-tdTomato (serving as a control vector) (tdTomato) or AAV8-hSyn-HA-GCN4-GS4-C5-WPRE-pA (GCN4-GS4-C5) 14 days prior to SNI. Von Frey testing revealed complete pain relief for AAV8-hSyn-HA-GCN4-GS4-C5-WPRE-pA treated mice at day 7, day 14 and day 28 post SNI, as compared to its own intact paw (contra), and as compared to control animals (Saline ipsi and tdTomato ipsi). Data are presented as mean±SEM and were analyzed using a 2-way ANOVA with Tukey's post-hoc test, F (30, 196)=6,511, p<0.0001.

FIG. 10: Intrathecal AAV8-hSyn-HA-GCN4-GS4-C5-WPRE-pA administered in mice exposed to the SNI model conferred expression towards the grey matter throughout the spinal cord at the lumbar vertebra level as visualized for positive immunoreactivity against the HA-tag (brighter white color).

FIG. 11: Effect of single amino acid substitutions in DAT C5 on binding affinity. Single amino acids substitutions in position Z₁-Z₅ of the sequence HWLKV were tested in fluorescence polarization binding in competition with fluorescently labelled DATCS. Data are given as fold change compared to the reference peptide HWLKV (set to 1).

FIG. 12: Competitive fluorescent polarization binding assay using A) the IETDV PBM targeting the PDZ1-2 of PSD-95 (PDZ class I PDZ domains), B) the RRTTPV binding motif targeting all three PDZ domains of PSD-95, and C) the YKQTSV binding motif primarily targeting PDZ3 of PSD-95. D) The PICK1 specific PBM class II ligand, GCN4p1-GS4-HWLKV, is not capable of binding PSD-95. The tested peptides (in A-D) contained an N-terminal Biotin and an Ahx linker.

FIG. 13: Circular dichroism of A) 7P14P-GS4-HWLKV (random coil) and GCN4p1-GS4-HWLKV (a-helix), and B) 7P14P-GS4-RRTTPV (random coil) and GCN4p1-GS4-RRTTPV (a-helix).

FIG. 14: Fluorescence polarization competition binding curves for the unlabelled peptides. A fixed concentration of PICK1 (0.15 μM) and PEG4 linked dimeric C5 as tracer (20 nM) was titrated with increasing concentration of the unlabelled peptides (DATCS, GCN4-GS4-HWLKW, GCN4-GS4-GS4, SSO10a-GS4-HWLKV). This caused a displacement of the fluorescently labelled molecule (tracer) with the unlabelled peptides, and gave rise to decrease in the polarization value (mP) as seen in the plot. Data expressed as mean±SEM (n=3). In the table below, determined affinity (K) values (M) from the ‘One site—Fit’ K curve (plot above) for the unlabelled peptides calculated in GraphPad Prism 7.0. The tested peptides, except DATCS, contained an N-terminal Biotin and an Ahx linker.

FIG. 15: Size exclusion chromatography of 40 μM PICK1 (A) in complex with 20 μM GCN4-GS4-HWLKV (B), 20 μM GCN4-GS4-GS4 (C), 20 μM SSO10a-GS4-HWLKV (D). The tested peptides, except PICK1, contained an N-terminal Biotin and an Ahx linker.

FIG. 16: Size exclusion chromatography of (A) Peptides GCN4-GS4-C5, 7P14P-GS4-C5 and GCN4-GS4-V-to-D at a concentration of 400 μM, (B) PICK1 (40 μM) and GCN4-GS4-C5 (400 μM), (C) PICK1 (40 μM) and 7P14P-GS4-C5 (400 μM), (D) PICK1 (40 μM) and GCN4-GS4-V-to-D (400 μM), (E) PICK1 (40 μM) and GCN4-GS4-C5 (20 μM).

FIG. 17: Both AAV8-hSyn-HA-GCN4p1-GS4-C5-WPREpA and AAV8-hSyn-HA-GCN4p1(7P14P)-GS4-C5-WPREpA efficiently relief pain in the CFA model of inflammatory pain. Data was analysed by two-way ANOVA (interaction: F(6,51)=4.203 p=0.0016; Time: F(3,51)=28.40 p<0.0001; Treatment: F(2,17)=11.12 p=0.0008). Post-Bonferroni analysis revealed significant pain relief at both day 2 and 5 post CFA injection (7P14P_{day2} p<0.0001; 7P14P_{day5} p=0.0003; GCN4_{day2} p=0.07; GCN4_{day5} p=0.0008), when comparing pain threshold to the tdTomato control. Furthermore, both treatments prevented latent sensitization in the CFA model of inflammatory pain. Data was analysed by two-way ANOVA (interaction: F(4,26)=5.237 p=0.0031; Time: F(2,26)=16.08 p<0.0001; Treatment: F(2,13)=5.662 p=0.017). Post-Bonferroni analysis revealed prevention of latent sensitization at 1 hour after naltrexone injection (7P14P_{1hrpostNTX} p<0.0001; GCN4_{1hrpostNTX} p<0.0001), when comparing pain threshold to the tdTomato control.

FIG. 18: Vector treatment with recombinant peptide targeting PICK1 can prevent a learning deficit in rats with amyloid-β pathology. Three weeks prior to the Morris Water maze testing session, the rats (n=20) received bilateral intraventricular deposit of amyloid-β. At the same time, the rats were either infused with AAV-EGFP (control, n=10) or AAV-GCN4-GS4-C5 (n=10) bilaterally into the hippocampus. Escape latency was tested in three consecutive swims (Swim 1-3).

FIG. 19: AAV vector delivery after SNI. AAV8-GCN4(7P14P)-GS4-C5 treatment after nerve injury completely reverse mechanical hyperalgesia in the mouse SNI model. Mice were injected intrathecally with AAV8-hSyn-tdTomato-WPREpA (n=10, serving as a control vector) or AAV8-hSyn-HA-GCN4p1(7P14P)-GS4-C5-WPREpA (n=10) two days after SNI. Von Frey testing revealed significant pain relief for AAV8-hSyn-HA-GCN4p1(7P14P)-GS4-C5 treated mice on day 7, day 14, day 21, day 28, day 35, day 43 post SNI as compared to control animals (tdTomato ipsi). Data are presented as mean±SEM and were analyzed using a 2-way ANOVA with Tukey's post-hoc test, F (24, 324)=8,644, p<0.0001.

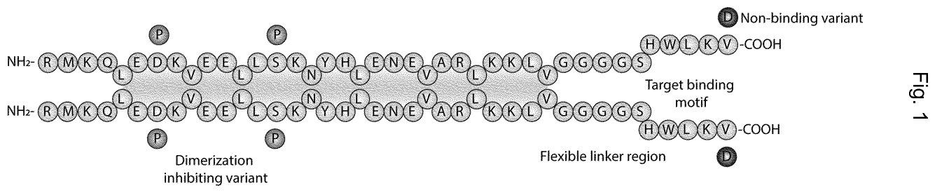

### DETAILED DESCRIPTION

### Definition of Abbreviations and Terms

Amino acids, that are proteinogenic are named herein using either its 1-letter or 3-letter code according to the recommendations from IUPAC, see for example http://www.chem.qmw.ac.uk/iupac. If nothing else is specified an amino acid may be of D or L-form. In the description (but not in the sequence listing) a 3-letter code starting with a capital letter indicates an amino acid of L-form, whereas a 3-letter code in small letters indicates an amino acid of D-form;

Hydrophobic amino acids, are amino acids having a hydrophobic side chain, examples of hydrophobic amino acids include alanine, isoleucine, leucine, methionine, phenylalanine, valine, proline and glycine.

CNS, central nervous system; AAV, adeno associated virus;

AAV1, Adeno-associated virus vectors serotype 1;

AAV2, Adeno-associated virus vectors serotype 2;

AAV5, Adeno-associated virus vectors serotype 5;

AAV8, Adeno-associated virus vectors serotype 8;

AAV9, Adeno-associated virus vectors serotyped with AAV9 capsid;

PDZ, acronym combining the first letters of the first three proteins discovered to share the domain Postsynaptic density protein-95 (PSD-95), Drosophila homologue discs large tumor suppressor (DIgA) and Zonula occludens-1 protein (zo-1). PDZ domains are common structural domains of 80-90 amino-acids found in signaling proteins. Proteins containing PDZ domains often play a key role in anchoring receptor proteins in the membrane to cytoskeletal components.

GS, glycine or glycine serine linker

hSyn, Human synapsin 1 gene promoter confers highly neuron-specific long-term transgene expression from an adenoviral vector.

WPRE, Woodchuck Hepatitis Virus (WHP) Posttranscriptional Regulatory Element. It is a DNA sequence that, when transcribed creates a tertiary structure enhancing expression and is commonly used in molecular biology to increase expression of genes delivered by viral vectors.

Amide bond is formed by a reaction between a carboxylic acid and an amine (and concomitant elimination of water). Where the reaction is between two amino acid residues, the bond formed as a result of the reaction is known as a peptide linkage (peptide bond).

Von Frey test assess touch sensitivity with von Frey filaments. These filaments are applied to the underside of the paw after the mouse has settled into a comfortable position within a restricted area that has a perforated floor. The filaments are calibrated to flex when the set force is applied to the paw. Filaments are presented in order of increasing stiffness, until a paw withdrawal is detected.

“Proteogenic” as used herein refers to the 20 amino acids that constitute all proteins that are naturally occurring.

Non-proteinogenic amino acids are amino acids which are not used in nature as building blocks for protein biosynthesis and are thereby to be clearly demarcated from the 20 proteinogenic amino acids.

The term ‘absent’ as used herein, e.g. “Z₁ is any proteogenic or non-proteogenic amino acid, preferably H, L, I, A or is absent” is to be understood as that the amino acid residues directly adjacent to the absent amino acid are directly linked to each other by a conventional amide bond.

The term ‘operably linked’ as used herein indicates that the polynucleotide sequence encoding one or more polypeptides of interest and transcriptional regulatory sequences are connected in such a way as to permit expression of the polynucleotide sequence when introduced into a cell.

The term “polypeptide” as used herein refers to a molecule comprising at least two amino acids. The amino acids may be natural or synthetic. The term “polypeptide” is also intended to include proteins, i.e. functional biomolecules comprising at least one polypeptide; when comprising at least two polypeptides, these may form complexes, be covalently linked or may be non-covalently linked. The polypeptides in a protein can be glycosylated and/or lipidated and/or comprise prosthetic groups.

The term ‘disorder’ used herein refers to a disease or medical problem, and is an abnormal condition of an organism that impairs bodily functions, associated with specific symptoms and signs.

The term ‘polynucleotide’ used herein refers to a molecule which is an organic polymer molecule composed of nucleotide monomers covalently bonded in a chain. A “polynucleotide” as used herein refers to a molecule comprising at least two nucleic acids. The nucleic acids may be naturally occurring or modified.

The term ‘promoter’ used herein refers to a region of DNA that facilitates the transcription of a particular gene. Promoters are typically located near the genes they regulate, on the same strand and upstream.

The term ‘medicament’ refers to any therapeutic or prophylactic agent which may be used in the treatment (including the prevention, diagnosis, alleviation, or cure) of a malady, affliction, condition, disease or injury in a patient.

The NMDA receptor refers to the N-methyl-D-aspartate receptor (also known as the NMDA receptor or NMDAR) and is a glutamate receptor and ion channel protein found in nerve cells. The NMDA receptor is one of three types of ionotropic glutamate receptors.

### Structure of PDZ Domain Inhibitors

The polynucleotide of the present invention encodes a PDZ domain inhibitor, such as a PICK1 inhibitor molecule which is capable of self-assembling into homodimers (Example 1) which may induce a tetrameric complex of PICK1 (Example 2) and obtain an affinity for the PDZ domain of e.g. PICK1 in the nanomolar range (Example 3). The polypeptide coded by the polynucleotide sequence can be expressed by an AAV vector in cells defined by site of injection and promoter specificity (Example 4). Upon injection the polypeptide will alleviate symptoms of pain in the spared nerve injury (SNI) model (Example 5) and Complete Freund's Adjuvants (CFA) model (Example 10).

The invention provides a polynucleotide comprising a sequence encoding upon expression a polypeptide comprising:

a) a first polypeptide part comprising or consisting of an amino acid sequence capable of forming a dimer; and
b) a second polypeptide part comprising or consisting of an amino acid sequence selected from the group consisting of Class I PDZ domains binding motifs (PBM), Class II PBM and Class III PBM,
wherein the first and the second polypeptides are optionally connected operably linked via a linker.

The term “capable of forming a dimer” refers to the ability of the first polypeptide part of said polypeptide to interact with a first polypeptide part of a second polypeptide and form a dimer, such as a homodimer. Such dimer may for instance be observed by analysis of the polypeptide by size exclusion chromatography (SEC), such as by the SEC method as described in Examples 2, 8, and 9 of the present disclosure. Thus, the polynucleotide of the present disclosure may provide a monomeric polypeptide upon expression, which is capable of interacting with a second polypeptide to form a dimer, such as a homodimer. The interaction of the two polypeptides may be facilitated via interaction of the two first polypeptide parts having an alpha helical secondary structure, such as an amphipathic helix. Such interaction between two alpha helical first peptide parts may form a coiled coil interaction. In one embodiment, the first polypeptide parts of the two polypeptides capable of forming a dimer has a low alpha helical content, such as determined by circular dichroism. The interaction between monomers to form a dimer may be facilitated by electrostatic interactions, such as hydrophobic interactions, salt-bridges and/or hydrogen bonding.

The invention further provides a polynucleotide comprising a sequence encoding upon expression a polypeptide comprising:

a) a first polypeptide part comprising or consisting of an amino acid sequence capable of forming an amphipathic helix; and
b) a second polypeptide part comprising or consisting of an amino acid sequence selected from the group consisting of Class I PDZ domains binding motifs (PBM), Class II PBM and Class III PBM,
wherein the first and the second polypeptides are optionally operably linked via a linker.

In one embodiment, the second polypeptide part is comprising or consisting of an amino acid sequence selected from the group consisting of Class I PDZ domains binding motifs (PBM), Class II PBM and Class III PBM.

In one embodiment, the second polypeptide is consisting of or comprising a sequence selected from the group consisting of ¥ and ϕ-¥-Ψ, wherein
Σ is Thr or Ser;
¥ is any proteinogenic amino acid;
Ψ is any hydrophobic amino acid; and
ϕ is Asp or Glu.

In one embodiment, the second polypeptide part is a Class I PBM comprising or consisting of a sequence of Σ-¥-Ψ, wherein is Thr or Ser, ¥ is any proteinogenic amino acid and Ψ is any hydrophobic amino acid, such as consisting of or comprising a sequence selected from the group consisting of IETDV (SEQ ID NO: 29), RRTTPV (SEQ ID NO: 32), and YKQTSV (SEQ ID NO: 35).

In one embodiment, the second polypeptide part is a Class III PBM comprising or consisting of a sequence of ϕ-¥-Ψ, wherein ϕ is Asp or Glu, ¥ is any proteinogenic amino acid and Ψ is any hydrophobic amino acid, such as consisting of or comprising a sequence selected from the group consisting of KVDSV (SEQ ID NO: 40), GKDYV (SEQ ID NO: 41), RKDYV (SEQ ID NO: 42), TAEMF (SEQ ID NO: 43) and QEDGA (SEQ ID NO: 44).

In one embodiment, the second polypeptide part is a Class II PBM comprising or consisting of a sequence of Ψ-¥-Ψ, wherein is any proteinogenic amino acid and Ψ is any hydrophobic amino acid, such as consisting of or comprising the sequence HWLKV (SEQ ID NO: 10).

In one embodiment, the second polypeptide part is comprising or consisting of an amino acid sequence of the general formula (II):

(SEQ ID NO: 11)
Z₁Z₂Z₃Z₄Z₅;

wherein:

Z₁ is a proteogenic or non-proteogenic amino acid, preferably H, L, V, I, A; or is absent;

Z₂ is a proteogenic or non-proteogenic amino acid, preferably W, F, T, S; or is absent;

Z₃ is a proteogenic or non-proteogenic amino acid, preferably L, V, I, F, A, Y;

Z₄ is a proteogenic or non-proteogenic amino acid, preferably K, R, T, S; and

Z₅ is V, I, L or C.

In one embodiment, the second polypeptide part is comprising or consisting of an amino acid sequence of the general formula (II):

(SEQ ID NO: 22)
Z₁Z₂Z₃Z₄Z₅;

wherein:

Z₁ is H, L, V, I, A; or is absent;

Z₂ is W, F, T, S; or is absent;

Z₃ is L, V, I, F, A, Y;

Z₄ K, R, T, S; and

Z₅ is V, I, L or C.

In one embodiment, the second polypeptide part is comprising or consisting of an amino acid sequence of the general formula (II):

(SEQ ID NO: 23)
Z₁Z₂Z₃Z₄Z₅;

wherein:

Z₁ is H, L, V, I, A;

Z₂ is W, F, T, 5;

Z₃ is L, V, I, F, A, Y;

Z₄ K, R, T, S; and

Z₅ is V, I, L or C.

In one embodiment, the second polypeptide part is comprising or consisting of an amino acid sequence of the general formula (II):

(SEQ ID NO: 24)
Z₁Z₂Z₃Z₄Z₅;

wherein:

Z₁ is H, V, I, A;

Z₂ is W, F, 5;

Z₃ is L, V, I;

Z₄ K, R, S; and

Z₅ is V, or C.

In one embodiment, the second polypeptide part is comprising or consisting of an amino acid sequence of the general formula (II):

(SEQ ID NO: 25)
Z₁Z₂Z₃Z₄Z₅;

wherein:

Z₁ is H, V, A;

Z₂ is W or S;

Z₃ is L, V, I;

Z₄ K or R; and

Z₅ is V.

In one embodiment, the second polypeptide part is selected from the group consisting of HWLKV, IETDV, RRTTPV, and YKQTSV.

In one embodiment, the second polypeptide part is selected from the group consisting of IETDV, RRTTPV, and YKQTSV.

In one embodiment, the second polypeptide part HWLKV.

In one embodiment, the first polypeptide part is comprising or consisting of an amino acid sequence capable of forming a dimer.

In one embodiment, the first polypeptide part is selected from the group consisting of GCN4p1 (SEQ ID NO: 8), GCN4p1(7P14P) (SEQ ID NO: 27), Atg16, MDV1 and SSO10a (SEQ ID NO: 45),

In one embodiment, the first polypeptide part is selected from the group consisting of GCN4p1, GCN4p1(7P14P), and SSO10a.

In one embodiment, the first polypeptide part is GCN4p1 (SEQ ID NO: 8).

In one embodiment, the first polypeptide part is GCN4p1(7P14P) (SEQ ID NO: 27).

In one embodiment, the first polypeptide part has an amino acid sequence of the general formula (I):

(formula (I), SEQ ID NO: 12)
L-[X]6-L-[X]6-L-[X]6-L,

wherein X is individually selected from any proteinogenic or non-proteinogenic amino acid residue.

In one embodiment, the first polynucleotide part is capable of forming a leucine zipper.

In one embodiment, the first polypeptide comprises or consists of the amino acid sequence RMKQLEDKVEELLSKNYHLENEVARLKKLV (SEQ ID NO: 8).

In one embodiment, the first polypeptide part has an amino acid sequence of the general formula (I): L-[X]₆-L-[X]₆-L-[X]₆-L (formula (I), SEQ ID NO:12),

wherein X is individually selected from any proteinogenic or non-proteinogenic amino acid residue.

In one embodiment, the polynucleotide of the present disclosure comprises a sequence encoding upon expression:

a) a first polypeptide comprising or consisting of an amino acid sequence capable of forming a leucine zipper; and
b) a second polypeptide comprising or consisting of an amino acid sequence of the general formula (II):

(SEQ ID NO: 11)
Z₁Z₂Z₃Z₄Z₅;

wherein:
Z₁ is a proteogenic or non-proteogenic amino acid, preferably H, L, V, I, A;
or is absent;
Z₂ is a proteogenic or non-proteogenic amino acid, preferably W, F, S, T;
or is
absent;
Z₃ is a proteogenic or non-proteogenic amino acid, preferably L, V, I, F; A, Y;
Z₄ is a proteogenic or non-proteogenic amino acid, preferably K, R, S, T; and
Z₅ is V, I, L or C.

The invention provides a polynucleotide comprising a sequence encoding upon expression:

a) a first polypeptide comprising or consisting of an amino acid sequence of the general formula (I): L-[X]₆-L-[X]₆-L-[X]₆-L (SEQ ID NO:12), wherein X is individually selected from any proteinogenic or non-proteinogenic amino acid residue; and
b) a second polypeptide comprising or consisting of an amino acid sequence of the general formula (II):

(SEQ ID NO: 11)
Z₁Z₂Z₃Z₄Z₅;

wherein:
Z₁ is a proteogenic or non-proteogenic amino acid, preferably H, L,V, I, A;
or is absent;
Z₂ is a proteogenic or non-proteogenic amino acid, preferably W, F, S, T;
or is
absent;
Z₃ is a proteogenic or non-proteogenic amino acid, preferably L, V, I, F; A, Y;
Z₄ is a proteogenic or non-proteogenic amino acid, preferably K, R, S, T; and
Z₅ is V, I, L or C.

In one embodiment, the first polypeptide further comprises any proteinogenic or non-proteinogenic amino acid [X], conjugated to the N- and/or C-terminus of the sequence of formula (I).

X as used herein should be understood as any proteogenic or non-proteogenic amino acid. Thus for example L-[X]₆-L-[X]₆-L-[X]₆-L could be L-VKAEHG-L-DKVEEQ-L-EVARAK-L (SEQ ID NO: 47). According to the general formula (I), there are six proteogenic or non-proteogenic amino acids between the amino acids L.

In one embodiment, the first polypeptide comprises the encoding protein sequence of the leucine zipper of GCN4 Accession number Q877C4, called p1GCN4, devoid of the DNA binding domain of GCN4. The GCN4 is normally found in yeast, where it serves as a bZIP transcription factor. The p1GCN4 sequence encodes seven repeats of amino acids forming an amphipathic a-helix with two surfaces along its lengths. The hydrophobic face dimerizes with itself, thereby forming a stable homodimeric structure.

In one embodiment, the leucine zipper is not homologous to a mammalian leucine zipper.

In one embodiment, the second polypeptide comprises or consists of the amino acid sequence of HWLKV (SEQ ID NO: 10).

In one embodiment, the first polypeptide part and the second polypeptide part is directly linked via an amide bond formed between the C-terminal carboxylic acid of one polypeptide and the N-terminal amine of the other polypeptide.

In one embodiment, the first polypeptide is operably linked to the second polypeptide, such as wherein the first polypeptide is linked to the second polypeptide via a polypeptide linker.

In one embodiment, the first polypeptide part is operably linked to the second polypeptide part via a linker, such as a peptide linker.

In one embodiment, the linker is a glycine serine (GS) linker. The glycine serine linker may be selected from the group consisting of GGS (gLinker2), GGGS (gLinker3, SEQ ID NO: 48), GGGGS (glinker4, SEQ ID NO: 9), GGGGSG (gLinker5, SEQ ID NO: 49), GGGGSGG (gLinker6, SEQ ID NO: 50).

In one embodiment, the linker is GGGGS (glinker4, SEQ ID NO: 9).

In one embodiment, the linker comprises 1 to 12 repeats of the GS linker, such as for example 1 to 12 repeats of the GGGGS moieties (N=1-12).

In yet another embodiment, the polypeptide linker comprises 3 to 12 repeats of the GGGGS moieties (N=3-12).

In one embodiment, the polypeptide linker is attached to the C-terminus of the first polypeptide.

In one embodiment, the polypeptide linker is a glycine rich linker. By glycine rich linker, it is understood that the linker comprise or consists of a plurality of amino acids G. In another embodiment, the polypeptide linker comprises or consists of the amino acid sequence of SEQ ID NO: 9.

In one embodiment, the first polypeptide part is positioned N-terminal to the second polypeptide part.

In one embodiment, the polypeptide further comprises a cell penetrating peptide (CPP).

In one embodiment, the CPP is connected to the polypeptide via a linker, such as a polypeptide linker, such as a glycine serine linker.

In one embodiment, the CPP is positioned N-terminal to the first and the second polypeptide parts.

In one embodiment, the CPP is selected from the group consisting of TAT, retroinverso-D-TAT, polyarginine, TP10, PNT and MAP.

In one embodiment, the polynucleotide further comprises a promoter that permits high expression in neurons, such as for example dorsal spinal horn neurons. In a preferred embodiment, said promoter is neuron-specific. In a most preferred embodiment, said promoter is a human synapsin promoter. In another embodiment, the promoter is a human Synapsin1 promoter.

In one embodiment, the promoter is a promoter specific for mammalian cells. In a further embodiment, said mammalian cell is a neural cell. In yet a further embodiment, said neural cell is a neuron.

In another embodiment, said promoter is a constitutive promoter. In one embodiment, the constitutively active promoter is selected from the group consisting of CAG, CMV, human ubiquitin C, RSV, EF-1alpha, NSE, SV40, Mt1.

In another embodiment, the promoter is an inducible promoter. In one embodiment, the inducible promoter is selected from the group consisting of Tet-On, Tet-Off, Mo-MLV-LTR, Mx1, progesterone, RU486 and Rapamycin-inducible promoter.

In one embodiment, the said promoter is an activity-dependent promoter. The said activity-dependent promoter may be selected from the group consisting of cFos, Arc, Npas4, Egr1 promoters.

In one embodiment, the promoter is Robust Activity Marking (RAM) promoter. This promoter is described by Sorensen et al., 2016, A robust activity marking system for exploring active neuronal ensembles. eLife 2016; 5:e13918s.

In another embodiment, the polynucleotide sequence of the present invention is regulated by a post-transcriptional regulatory element. In a preferred embodiment, said regulatory element is a Woodchuck hepatitis virus post-transcriptional regulatory element.

### The Recombinant Expression Vector

The present invention provides a recombinant expression vector comprising the said polynucleotide.

Broadly, gene therapy seeks to transfer new genetic material to the cells of a patient with resulting therapeutic benefit to the patient. Such benefits include treatment or prophylaxis of a broad range of diseases, disorders and other conditions.

Gene therapy may be classified into two distinct types: germ line gene therapy, wherein genetic material is transferred into germ cells and will thus be heritable, and somatic gene therapy, wherein genetic material is transferred into somatic cells and will thus not be heritable.

In one embodiment, the expression vector is selected from the group consisting of RNA based vectors, DNA based vectors, lipid based vectors, polymer based vectors and colloidal gold particles.

In another embodiment, the vector is a viral vector. The viral vector can be a virally derived DNA vector or a virally derived RNA vector.

Viral vectors are useful tools for delivering genetic material into a host organism. Viruses useful as gene transfer vectors include papovavirus, adenovirus, vaccinia virus, adeno-associated virus (AAV), herpes virus, and retroviruses, such as lentivirus, HIV, SIV, FIV, EIAV, MoMLV.

Thus, in one embodiment, The expression viral vector is selected from the group consisting of adenoviruses, recombinant adeno-associated viruses (rAAV), retroviruses, lentiviruses, adeno-associated viruses, herpesviruses, vaccinia viruses, foamy viruses, cytomegaloviruses, Semliki forest virus, poxviruses, RNA virus vector and DNA virus vector.

Preferred viruses for treatment of disorders of the central nervous system are lentiviruses and adeno-associated viruses. Both types of viruses can integrate into the genome without cell divisions, and both types have been tested in pre-clinical animal studies for indications in the nervous system, in particular in the central nervous system.

A preferred type of viral vector is the AAV. AAV is interesting in gene therapy due to a number of features. Chief amongst these is the wild-type virus' apparent lack of pathogenicity and that it can also infect non-dividing cells. The wild-type AAV genome integrates most frequently into a specific site (designated AAVS1) in the human chromosome 19, while random incorporations into the genome take place with a negligible frequency. The feature makes it somewhat more predictable than retroviruses, which present the threats of a random insertion and of mutagenesis. With AAVs as gene therapy vectors, however, this integrative capacity can be eliminated by removal of the rep and cap from the DNA of the vector. As the rep and cap genes have no functional value in a replication deficient viral vector, they can be eliminated from the vector genome. In the place of these wild-type AAV genes, the desired gene(s) together with a promoter to drive transcription of the gene can be inserted between the inverted terminal repeats (ITR). The ITRs are important for the viral vector packaging of the vector DNA and aids in concatamer formation in the nucleus after the single-stranded vector DNA is converted by host cell DNA polymerase complexes into double-stranded DNA.

AAV-based gene therapy vectors can form episomal concatamers in the host cell nucleus. In non-dividing cells, these concatamers can remain intact for the life of the host cell. In dividing cells, AAV DNA can be lost through cell division, since the episomal DNA is not replicated along with the host cell DNA. Random integration of AAV DNA into the host genome is low but may be detectable. AAV's present low immunogenicity seemingly restricted to the generation of neutralizing antibodies, while they induce no clearly-defined cytotoxic response. These features, along with the ability to infect quiescent cells, present some of their advantages over adenoviruses as vectors for the human gene therapy. These features make AAV an attractive candidate for creating viral vectors for gene therapy in the central nervous system (CNS).

At least 11 serotypes of the AAV presently exist. Serotype 2 has been most extensively investigated, and AAV2 presents natural tropism towards e.g., skeletal muscles, vascular smooth muscle cells, hepatocytes and in particular neurons. However, other serotypes have proved effective as tolls for gene therapy; for instance AAV6, AAV7 and AAV8.

The humoral immunity instigated by infection with the wild type AAV is thought to be a very common event. The associated neutralizing activity limits the usefulness of the most commonly used serotype AAV2 in certain applications. Accordingly the majority of clinical trials currently underway into the brain involve delivery of AAV2, a relatively immunologically privileged organ.

In addition to using different serotypes of the AAV, it is possible to combine different serotypes, such as using the plasmid of one serotype packaged in the capsid of another serotype.

In one embodiment the adeno associated vector (AAV) vector of the present invention is an AAV2 vector.

In a further embodiment the AAV2 vector is packaged in an AAV capsid other than an AAV2 capsid.

In yet a further embodiment the AAV2 vector is packed in an AAV5 capsid.

In a preferred embodiment, the adeno associated vector (AAV) vector of the present invention is an AAV8 vector. In a further embodiment the AAV8 vector is packaged in an AAV capsid other than an AAV8 capsid.

In one embodiment the adeno associated vector (AAV) plasmid of the present invention is packaged as an AAV2 vector.

In a further embodiment the AAV plasmid is packaged as an AAV capsid other than an AAV2 capsid.

In yet a further embodiment the AAV plasmid is packed as an AAV5 vector.

In a preferred embodiment, the adeno associated vector (AAV) vector of the present invention is an AAV8 vector. In a further embodiment the AAV plasmid is packaged in an AAV capsid other than an AAV8 capsid.

In yet another embodiment, the AAV plasmid is packaged as an AAV9 vector.

In one embodiment, the vector based on AAV vectors can be of any serotype modified to express altered or novel coat proteins.

In one embodiment, the vector is based on any AAV serotype identified in humans, non-human primates, other mammalian species, or chimeric versions thereof.

AAV vectors can be prepared using two major principles, transfection of human cell line monolayer culture or free floating insect cells. Monolayer cell cultures are transfected through calcium phosphate precipitation, lipofection or other means with a mix of two or three plasmid preparations containing a transfer plasmid with the vector genome and one or two helper plasmids containing the necessary genes for vector capsid synthesis. For insect sell cultures, this process is normally replaced by transfection of the cells using baculovirus constructs that contain the same functions. The cells, supernatant or both are then collected for purification and concentration of the vector. This can be achieved through any combination of caesium chloride or iodixanol gradient purification, ion exchange chromatography, gel filtration and affinity chromatography and ultracentrifugation. Any methods for preparation and delivery to the CNS of AAV known in the art can be used.

Accordingly, in a main aspect, the present invention relates to a recombinant expression vector comprising the said polynucleotide sequence.

In one embodiment, the recombinant vector and its resulting oligopeptide is termed HA-GCN4-GS4-C5, and can be encoded by a viral vector if inserted downstream of a promoter.

In one embodiment, the recombinant vector encodes a polypeptide termed: GCN4-GS4-C5 (SEQ ID NO: 6), GCN4(7P14P)-GS4-C5 (SEQ ID NO: 28), GCN4-GS4-IETDV (SEQ ID NO: 30), GCN4(7P14P)-GS4-IETDV (SEQ ID NO: 31), GCN4-GS4-RRTTPV (SEQ ID NO: 33), GCN4(7P14P)-GS4-RRTTPV (SEQ ID NO: 34), GCN4-GS4-YKQTSV (SEQ ID NO: 36), GCN4(7P14P)-GS4-YKQTSV (SEQ ID NO: 37), or SSO10A-GS4-C5 (SEQ ID NO: 46).

After delivery by a viral vector into living cell, the synthetic encoded DNA sequence is first transcribed, then translated into a single-stranded oligopeptide (monomer) which is capable of assembling into a dimeric peptide. This is due to the genetic engineering and insertion of the first polypeptide such as an element derived from the leucine zipper of the yeast bZIP transcription factor GCN4 conjugated to the second polypeptide which constitutes the PDZ domain binding motif (PBM), such as for PICK1.

In one embodiment, the vector comprises or consists of a polynucleotide sequence selected from the group consisting of SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 60.

In one embodiment, the vector comprises or consists of the polynucleotide sequence SEQ ID NO: 1.

In one embodiment, the vector is functional in mammalian cells. In a preferred embodiment, the vector is functional in a neural cell. In another embodiment, the vector is functional in a neuron.

The present invention provides a polypeptide encoded by the said polynucleotide or the expression vector. In one embodiment, the polypeptide is in the form of a multimer. In another embodiment, the polypeptide is in the form of a dimer, a tetramer or a hexamer. In yet another embodiment, the polypeptide is in the form of a dendrimer. The present invention provides a recombinant host cell comprising the said expression vector, the said polynucleotide or the said polypeptide.

### Protein-Protein Interactions of PDZ Domains

PDZ domains are known to increase the specificity and efficiency of intracellular communication networks downstream of receptor activation by facilitating several protein-protein interactions (PPIs). PDZ domains may be found in multidomain scaffold and anchoring proteins involved in trafficking, recruiting, and assembling of intracellular enzymes and membrane receptors into signal-transduction complexes.

In one embodiment, the second polypeptide of the present invention binds to a PDZ domain.

In some embodiment, binding of the polypeptide encoded by the polynucleotide of the present disclosure, result in dimerization of the PDZ domain to which it binds. For example, the polypeptide may bind to PDZ domains of two separate proteins, thereby bringing the two proteins together to form a dimer. In one embodiment, the PDZ domains are inhibited by this dimer formation. In one embodiment, the PDZ domain is PICK1 which has a dimer configuration. Thus, in one embodiment, binding of the polypeptide encoded by the polynucleotide of the present disclosure to PICK1, result in formation of dimers of dimers, such as tetramer formation of PICK1.

In a separate embodiment, the polypeptide encoded by the polynucleotide does not result in dimerization of the PDZ domain. Thus, in one embodiment, the polypeptide encoded by the polynucleotide of the present disclosure provides inhibition of the PDZ domain in the absence of dimerization of the PDZ domain.

In another embodiment, the second polypeptide is capable of inhibiting the protein-protein interaction of a PDZ domain and its respective binding partner.

In one embodiment, the second polypeptide is capable of inhibiting a protein-protein interaction with the PDZ domain, such as the interaction between AMPAR and PICK1, between cytosolic kinases and PICK1, between synaptic scaffold proteins and PICK1, between membrane embedded proteins and PICK1, between NMDAR and PSD-95, between membrane embedded proteins and PSD-95, or between synaptic scaffold proteins and PSD-95.

the second polypeptide inhibits the PDZ domain, such as inhibits PICK1, PSD-95, Shank1, Shank2, Shank3, nNOS, syntenin, GRIP, MAGI I, MAGI2, MAGI3, PSD-93, DLG1, ZO-1, Frizzled, PAR3, or PARE, Mint1, or CASK.

In one embodiment, the polypeptide has a K for the PDZ domain below 25 μM, such as below 10 μM, such as below 5 μM, such as below 1 μM, such as below 800 nM, such as below 600 nM, such as below 400 nM, such as below 300 nM, such as below 200 nM, such as below 160 nM, such as below 150 nM, such as below 140 nM, such as below 130 nM, such as below 120 nM, such as below 110 nM, such as below 100 nM, such as below 90 nM, such as below 80 nM, such as below 70 nM. K may be determined by the method as disclosed in Examples 3, 6, 7 and 8.

### AMPAR-PICK1 Interaction

AMPARs are usually only permeable to monovalent cations (i.e. Na+ and K+) due to presence of the GluA2 subunit in the receptor complex. A specific type of plasticity involving strong and sustained depolarization, however, results in a switch to AMPARs, excluding the GluA2 subunit, with increased conductance and Ca²⁺ permeability (CP-AMPARs) in several types of synapses. Since the AMPARs are readily activated, this switch renders the synapse hypersensitive with respect to both Na+ and Ca²⁺ calcium influx stimulated by glutamate. This plasticity plays a central pathophysiological role in development of addiction, initially in midbrain dopaminergic neurons and subsequently, as the addiction process progresses, also in medium spiny neurons, where it underlies cocaine craving. A similar process is involved in the development of neuropathic pain, first in the dorsal horn and subsequently and conceivably, also in the neurons in thalamus and sensory cortex. Finally, CP-AMPARs are also expressed in hippocampal neurons after ischemia and as such the process rather appears to be a maladaptive type of plasticity in response to abnormal levels of glutamate in the synapse. Mechanistically, expression of CP-AMPARs involves an initial PICK1 dependent down-regulation of GluA2 containing AMPARs, which is mediated by the interaction between the PICK1 PDZ domain and the C-terminus of theGluA2 subunit of the AMPARs. The downregulation of GluA2 containing AMPARs is in part regulated by phosphorylation of the AMPAR C-terminal regions by cytosolic kinases; these phosphorylations are also regulated by kinase binding to PICK1.

This in turn allows for insertion of GluA2 lacking receptors in the synapse rendering the synapse Ca²⁺-permeable and hypersensitive.

The second polypeptide of the present invention binds to a PDZ domain. Thus, in one embodiment, the second polypeptide binds to PICK1. In another embodiment, the second polypeptide is capable of inhibiting the protein-protein interaction between AMPAR and PICK1 described above. This can thus prevent PICK1 from down-regulating GluA2 and prevent CP-AMPARs formation thereby preventing a maladaptive type of plasticity in response to abnormal levels of glutamate in the synapse. This in turn can prevent for example neuropathic pain.

In yet another embodiment, the second polypeptide inhibits PICK1. The inhibition has the same purpose as the second polypeptide binding to a PDZ domain namely preventing interaction with AMPA receptors.

In yet another embodiment, the second polypeptide inhibits PICK1. The inhibition has the same purpose as the second polypeptide binding to a PDZ domain namely preventing interaction with cytosolic kinases. This can thus prevent PICK1 from down-regulating GluA2 and prevent CP-AMPARs formation thereby preventing a maladaptive type of plasticity in response to abnormal levels of glutamate in the synapse. This in turn can prevent for example neuropathic pain.

The second polypeptide has a high affinity for PICK1 compared to endogenous peptide ligands. In one embodiment, said second polypeptide has a Ki for PICK1 below 170 nM, such as below to 160 nM, for example below 150 nM, such as below to 140 nM, for example below 130 nM, such as below to 120 nM, for example below 110 nM, such as below to 100 nM, such as below to 90 nM, such as below to 80 nM, for example below 70 nM, such as below 60 nM, such as below to 50 nM, for example below 40 nM, such as below 30 nM, for example below 20 nM, for example below 10 nM. In one embodiment, the AMPAR is comprised in a cell.

### Detection

In one embodiment, the first polypeptide further comprises a tag. Conventional tags known to those of ordinary skill in the art for detection can be used such as a fluorescent protein or an antibody tag. The detectable tag can be for example GFP, enhanced GFP (EGFP) or TdTomato and the antibody Tag can be for example HA, c-myc, His-tag or biotin.

In one embodiment, the tag is conjugated to the N-terminus of the first polypeptide. In one embodiment, an HA-tag and a GS linker is added to the N terminus of the first polypeptide, for identification and tracking purposes. In one embodiment, the first polypeptide is further conjugated to biotin. In another embodiment, the biotin is attached to the N-terminus of the first polypeptide.

In one embodiment, the tags can be operably linked to the N-terminus of the first polypeptide by a linker sequence. For example, a linker sequence comprises or consists of the amino acid sequence GS. In another embodiment, biotin can be operably linked to the N-terminus of the first polypeptide by a linker sequence such as Ahx linker.

### Treatment

The present invention provides a composition comprising the polynucleotide, the expression vector or the polypeptide as disclosed herein. In another embodiment, the composition is a pharmaceutical composition. Such a composition typically contains the PDZ inhibitor, such as the PICK1 inhibitor of the invention in a pharmaceutically accepted carrier.

The present invention provides the polynucleotide, the expression vector, the polypeptide, the cell, and/or the composition as described herein for use as a medicament.

The present invention provides the polynucleotide, the expression vector, the polypeptide, the cell, and/or the composition as described herein for use in treatment of a disease and/or disorder associated with maladaptive plasticity and/or transmission.

The present invention further provides the polynucleotide, the expression vector, the polypeptide, the cell, and/or the composition as described herein for the manufacture of a medicament for the treatment of diseases and/or disorders associated with maladaptive plasticity and/or transmission.

The present invention further provides a method of treatment or prevention of a disease and/or disorder associated with maladaptive plasticity and/or transmission in a subject in need thereof, comprising administering a therapeutically effective amount of the polynucleotide, the expression vector, the polypeptide, the cell, and/or the composition as described herein.

The present invention further provides a method of treatment or prevention of pain in a subject in need thereof, comprising administering a therapeutically effective amount of the polynucleotide, the expression vector, the polypeptide, the cell, and/or the composition as described herein.

By therapeutically effective amount it is understood that the amount of the said polynucleotide, expression vector, polypeptide, cell and composition of the present invention is administered in sufficient quantity to achieve the intended purpose, such as, in this case, to treat disease and/or disorders associated with maladaptive plasticity and/or transmission in the patient.

The present invention provides the use of the said polynucleotide or the said recombinant expression vector or the said pharmaceutical composition for the manufacture of a medicament for the treatment of a disease and/or disorder associated with maladaptive plasticity and/or transmission.

The term “treatment” as used herein refers to any kind of treatment, including preventive, ameliorating/palliative or curative treatment. Treatment may thus result in the prevention, decrease and/or amelioration/palliation of causes and/or symptoms of diseases and/or disorders of maladaptive plasticity and/or transmission.

### Diseases and Disorders

PDZ-containing proteins are known to play an important role in cancer, from tumor formation to metastasis, especially through canonical interactions of their PDZ domains in signaling pathways. In fact, 145 of 151 PDZ domain proteins have been suggested to be associated with cancers. Validated drug targets include Scribbled, Syntenin and Disheveled.

A large number of PDZ domain-containing proteins are associated with neurological disorders. Among others, regulating synaptic membrane exocytosis protein 1 (RIMS1), partitioning defective 3 homolog B (PARD3B), peripheral plasma membrane protein CASK, and disks large homolog 4 (DLG4, PSD-95) are associated with neurodevelopmental disorders, which are central nervous system development disorders with different manifestations. Validated drug targets include PSD95, PICK1 and SHANK.

The interaction between CFTR and CAL has been studied due to its role in cystic fibrosis, where CAL promotes degradation of CFTR in lysozymes, and thereby reduces the amount of CFTR expressed at the surface.

The present invention provides the polynucleotide, the expression vector, the polypeptide, the cell, and/or the composition as described herein for use in treatment of a disease and/or disorder associated with maladaptive plasticity and/or transmission. In diseased states, such as ischemia after stroke, neuropathic pain and addiction, abnormal synaptic stimulation causes maladaptive plasticity leading to hyper-sensitization of glutamatergic synapses through expression of calcium permeable (CP) AMPA-type glutamate receptors (CP-AMPARs).

AMPA-type glutamate receptors (AMPARs) are, in contrast to NMDA-type glutamate receptors (NMDARs), usually only permeable to monovalent cations (i.e. Na+ and K+) due to presence of GluA2 subunits in the tetrameric receptor complex. Plasticity changes in response to a strong and sustained depolarization, however, result in a switch to AMPARs with increased conductance and Ca2+ permeability (CP-AMPARs) in several types of synapses and this switch renders the synapse hypersensitive. Mechanistically, expression of CP-AMPARs involves an initial PICK1-dependent down-regulation of GluA2 containing AMPARs, which is mediated by the interaction between the PICK1 PDZ domain and the C-terminus of the GluA2 subunit of the AMPARs. This in turn allows for insertion of GluA2 lacking receptors in the synapse (Slot hypothesis) rendering the synapse Ca2+-permeable and hypersensitive.

CP-AMPARs are critically involved in the mediating craving after withdrawal from cocaine self-administration in rats (Conrad et al 2008). PICK1 has been implicated in the expression of CP-AMPAR in the VTA dopaminergic neurons in midbrain and in nucleus accumbens during development of cocaine craving (Luscher et al 2011 and Wolf et al 2010) suggesting PICK1 as a target in cocaine addiction.

Upregulation of AMPA-type glutamate receptors (AMPARs) in the dorsal horn (DH) neurons causes central sensitization, a specific form of synaptic plasticity in the DH sustainable for a long period of time (Woolf et al 2000 and Ji et al 2003). Moreover, both peripheral inflammatory pain and nerve injury induced pain, cause upregulation of Ca2+-permeable AMPARs (CP-AMPARs) (Vikman et al 2008, Gangadharan et al 2011 and Chen et al 2013). Initial evidence for a role of PICK1 in neuropathic pain came from Garry et al 2003 demonstrating that peptide inhibitors of PICK1 alleviated pain induced by chronic constriction injury (CCI). Subsequently, it was demonstrated the shRNA mediated knock down of PICK1 alleviated complete Freud's adjuvans (CFA) induced inflammatory pain and it was found that PICK1 knock-out mice completely fail to develop pain in response to spinal nerve ligation (SNL) (Wang et al 2011 and Atianjoh et al 2010). Indeed, i.t. administration of the polynucleotides of the present disclosure reduces mechanical allodynia in a model of neuropathic pain (SNI model—example 5) and inflammatory pain (CFA model—example 10).

Both TDP-43 pathology and failure of RNA editing of the AMPA receptor subunit GluA2, are etiology-linked molecular abnormalities that concomitantly occur in the motor neurons of the majority of patients with amyotrophic lateral sclerosis (ALS). Pain symptoms in a mouse model with conditional knock-out of the RNA editing enzyme adenosine deaminase acting on RNA 2 (ADAR2) are relieved by the AMPAR antagonist perampanel, suggesting a likely symptomatic relief by the polynucleotides or polypeptides of the present disclosure.

Given the effect of the polynucleotides or polypeptides of the present disclosure on pain and predicted effect on addiction, we expect also good efficacy of virally encoded PICK1 inhibitors on patient with comorbidity e.g. pain patients with opioid addiction.

Similar central sensitization is thought to underlie the allodynia in hyperalgesic priming, which serves as an experimental model for lower back pain and migraine (Kandasamy et al 2015). Similarly, the etiology for tinnitus hold several parallels with neuropathic pain including central sensitization (Vanneste et al 2019, Peker et al 2016 and Moller et al 2007).

A role for PICK1 in the surface stabilization/insertion of CP-AMPARs has been described for oxygen-glucose depletion in cultured hippocampal neurons (Clem et al 2010 and Dixon et al 2009). This evokes PICK1 as a putative target in the protection of neural death after ischemic insult.

Loss of PICK1 has been demonstrated to protect neurons in vitro and in vivo against spine loss in response to amyloid beta (Marcotte et al 2018 and Alfonso et al 2014). Consequently, PICK1 is a putative target for symptomatic and perhaps preventive treatment of Alzheimer's disease, as demonstrated in Example 11.

PICK1 interacts and inhibits the E3 ubiquitin ligase Parkin, which is involved in mitophagy. Parkin loss of function is associated with both sporadic and familial Parkinson's disease (PD). As a result, PICK1 KO mice are resistant to 1-methyl-4-phenyl-1,2,3,6-tetrahydropyridine (MPTP)-mediated toxicity (He et al 2018). Consequently, PICK1 is a putative target for symptomatic and perhaps preventive treatment of Parkinson's disease.

Overstimulation of glutamate receptors resulting in excessive intracellular calcium concentrations is a major cause of neuronal cell death in epilepsy. The GluR2 (GluA2) hypothesis states that following a neurological insult such as an epileptic seizure, the AMPA receptor subunit GluR2 protein is downregulated. This increases the likelihood of the formation of GluR2-lacking, calcium-permeable AMPA receptor which might further enhance the toxicity of the neurotransmitter, glutamate (Lorgen et al 2017).

PICK1 is overexpressed in tumor cells as compared to adjacent normal epithelia in breast, lung, gastric, colorectal, and ovarian cancer. As judged by immunostaining breast cancer tissue microarrays, high levels of PICK1 expression correlates with shortened span of overall survival. Accordingly, transfection of MDA-MB-231 cells with PICK1 siRNA decreased cell proliferation and colony formation in vitro and inhibited tumorigenicity in nude mice (Zhang et al 2010). Consequently, PICK1 is a putative target for cancer treatment and prognostics.

In one embodiment, the polynucleotide as disclosed herein is for use in the prophylaxis and/or treatment of neuropathic pain, drug addiction, amyotrophic lateral sclerosis, epilepsy, tinnitus, migraine, ischemia, Alzheimer's disease, and/or Parkinson's disease. In one embodiment, the polynucleotide as disclosed herein is for use in the prophylaxis and/or treatment of neuropathic pain, drug addiction, amyotrophic lateral sclerosis, epilepsy, tinnitus, and/or migraine. The drug addiction may be opioid addiction. In a preferred embodiment, the polynucleotide as disclosed herein is for use in the prophylaxis and/or treatment of neuropathic pain.

In another embodiment, the polynucleotide as disclosed herein is for use in the prophylaxis and/or treatment of pain in a subject. The pain can be inflammatory pain or neuropathic pain. The pain, to be treated, may be chronic pain, which may be chronic neuropathic pain or chronic inflammatory pain. The neuropathic pain may be induced by damage to the peripheral or central nervous system as a result of traumatic injury, surgery, or diseases such as diabetes or autoimmune disorders. The neuropathic pain may be induced by treatment with chemotherapy. Where pain persists, the condition is chronic neuropathic pain. Chronic inflammatory pain may be induced by inflammation after nerve injury, as well as being initiated by inflammation induced by alien matter, where mediators released by immune cells cause a sensitization of pain pathways, i.e. a ‘wind up’ of sensory neurons located in the spinal cord. Thus, an effective analgesic drug must be able to reach spinal cord tissue and find its target, in this case PICK1, in order to have a pain-relieving effect. Thereby, the polynucleotide must be able to pass the blood-brain barrier and/or blood-spinal cord barrier to be able to reach spinal cord tissue and/or be expressed in desirable cells e.g. neurons.

In yet another embodiment, the polynucleotide as disclosed herein is for use in the prophylaxis and/or treatment of drug addiction. The drug addiction may be opioid addiction or cocaine addiction. For example the opioid addiction may be morphine addiction.

In yet another embodiment, the polynucleotide as disclosed herein is for use in the prophylaxis and/or treatment of head injury.

In yet another embodiment, the polynucleotide as disclosed herein is for use in the prophylaxis and/or treatment of stroke or ischemia.

In yet another embodiment, the polynucleotide as disclosed herein is for use in the prophylaxis and/or treatment of Alzheimer's disease.

In yet another embodiment, the polynucleotide as disclosed herein is for use in the prophylaxis and/or treatment of Parkinson's disease.

In yet another embodiment, the polynucleotide as disclosed herein is for use in the prophylaxis and/or treatment of cancer, such as breast cancer.

In one embodiment, the pharmaceutical composition is for use in the prophylaxis and/or treatment of neuropathic pain, drug addiction, amyotrophic lateral sclerosis, epilepsy, tinnitus, migraine, ischemia, Alzheimer's disease, and/or Parkinson's disease.

In one embodiment, the present invention provides the expression vector, the polypeptide, the cell, and/or the composition as described herein for use in the prophylaxis and/or treatment of neuropathic pain, drug addiction, amyotrophic lateral sclerosis, epilepsy, hearing disorders (i.e. tinnitus) and migraine. The drug addiction may be opioid addiction. In another embodiment, the drug addiction may be cocaine addiction. The opioid addiction may be for example morphine addiction. In a preferred embodiment, the expression vector, the polypeptide, the cell, and/or the composition is for use in the prophylaxis and/or treatment of neuropathic pain. In another embodiment, the expression vector, the polypeptide, the cell, and/or the composition is for use in the prophylaxis and/or treatment of pain in a subject. The pain can be inflammatory pain or neuropathic pain. The pain, to be treated, may be chronic pain, which may be chronic neuropathic pain or chronic inflammatory pain. The neuropathic pain may be induced by damage to the peripheral or central nervous system as a result of traumatic injury, chemotherapy, surgery, or diseases such as diabetes or autoimmune disorders. Where pain persists the condition is chronic neuropathic pain. Chronic inflammatory pain may be induced by inflammation after nerve injury, as well as being initiated by inflammation induced by alien matter, where mediators released by immune cells cause a sensitization of pain pathways, i.e. a ‘wind up’ of sensory neurons located in the spinal cord. Thus, an effective analgesic drug must be able to reach or be expressed in spinal cord tissue and find its target, in this case the PICK1 inhibitor peptide, in order to have a pain-relieving effect.

Thus, in one embodiment, the disease and/or disorder associated with maladaptive plasticity is selected from the group consisting of neuropathic pain, drug addiction, ischemia, Alzheimer's disorder, Parkinson's disease, amyotrophic lateral sclerosis, hearing disorders (e.g. tinnitus), migraine and epilepsy.

In one embodiment, the disease and/or disorder associated with maladaptive plasticity is selected from the group consisting of neuropathic pain, drug addiction, ischemia, Alzheimer's disorder, Parkinson's disease, amyotrophic lateral sclerosis, hearing disorders (e.g. tinnitus), migraine, breast cancer and epilepsy.

Subjects at risk or presently suffering from the above disorders and diseases may be given either prophylactic treatment to reduce the risk of the disorder or disease onset or therapeutic treatment following the disorder or disease onset. The subject may be a mammalian or human patient.

### Administration

Conventional methods, known to those of ordinary skill in the art of medicine, can be used to administer compositions to the subject or patient.

Conventional methods, known to those of ordinary skill in the art of medicine, can be used to administer the pharmaceutical formulation(s) of the present invention to the patient. The pharmaceutical compositions of the present invention can be administered alone, or in combination with other therapeutic agents or interventions. Specifically, the compositions of the present invention may further comprise a plurality of agents of the present invention. The present invention further includes a method of providing prophylaxis and/or treatment of diseases and/or disorders associated with maladaptive plasticity or pain in a subject, comprising administering the above pharmaceutical composition to the subject in need thereof.

In one embodiment, the pharmaceutical composition of the present disclosure is administered prior to observing symptoms of a given indication, such as administered prior to injury for the treatment of pain. In one embodiment, the pharmaceutical composition of the present disclosure is administered after observing symptoms of a given indication, such as administered after injury for the treatment of pain.

### Items

1. A polynucleotide comprising a sequence encoding upon expression a polypeptide comprising:
   a) a first polypeptide part comprising or consisting of an amino acid sequence capable of forming a dimer; and
   b) a second polypeptide part comprising or consisting of an amino acid sequence selected from the group consisting of Class I PDZ domains binding motifs (PBM), Class II PBM and Class III PBM,
   wherein the first and the second polypeptides are optionally operably linked via a linker.
2. The polynucleotide according to item 1, wherein the second polypeptide is consisting of or comprising a sequence selected from the group consisting of Σ-¥-Ψ, Ψ-¥-Ψ, and ϕ-¥-Ψ, wherein
   Σ is Thr or Ser;
   ¥ is any proteinogenic amino acid;
   Ψ is any hydrophobic amino acid; and
   ϕ is Asp or Glu.
3. The polynucleotide according to any one of the preceding items, wherein the second polypeptide part is a Class I PBM comprising or consisting of a sequence of Σ-¥-Ψ, wherein Σ is Thr or Ser, ¥ is any proteinogenic amino acid and Ψ is any hydrophobic amino acid, such as consisting of or comprising a sequence selected from the group consisting of IETDV, RRTTPV, and YKQTSV.
4. The polynucleotide according to any one of the preceding items, wherein the second polypeptide part is a Class III PBM comprising or consisting of a sequence of ϕ-¥-Ψ, wherein ϕ is Asp or Glu, ¥ is any proteinogenic amino acid and Ψ is any hydrophobic amino acid, such as consisting of or comprising a sequence selected from the group consisting of KVDSV, GKDYV, RKDYV, TAEMF and QEDGA.
5. The polynucleotide according to any one of the preceding items, wherein the second polypeptide part is a Class II PBM comprising or consisting of a sequence Ψ-¥-Ψ, wherein is any proteinogenic amino acid and Ψ is any hydrophobic amino acid, such as consisting of or comprising the sequence HWLKV.
6. The polynucleotide according to any one of the preceding items, wherein the second polypeptide part is comprising or consisting of an amino acid sequence of the general formula (II):

(SEQ ID NO: 11)
Z₁Z₂Z₃Z₄Z₅;

wherein:
Z₁ is a proteogenic or non-proteogenic amino acid, preferably H, L, V, I, A; or is absent;
Z₂ is a proteogenic or non-proteogenic amino acid, preferably W, F, T, S;
or is absent;
Z₃ is a proteogenic or non-proteogenic amino acid, preferably L, V, I, F; A, Y;
Z₄ is a proteogenic or non-proteogenic amino acid, preferably K, R, T, S; and
Z₅ is V, I, L or C.
7. The polynucleotide according to any one of the preceding items, wherein the second polypeptide part is comprising or consisting of an amino acid sequence of the general formula (II):

(SEQ ID NO: X)
Z₁Z₂Z₃Z₄Z₅;

wherein:
Z₁ is H, L, V, I, A; or is absent;
Z₂ is W, F, T, S; or is absent;
Z₃ is L, V, I, F, A, Y;
Z₄ K, R, T, S; and
Z₅ is V, I, L or C.
8. The polynucleotide according to any one of the preceding items, wherein the second polypeptide part is comprising or consisting of an amino acid sequence of the general formula (II):

(SEQ ID NO: X)
Z₁Z₂Z₃Z₄Z₅;

wherein:
Z₁ is H, L, V, I, A;
Z₂ is W, F, T, S;
Z₃ is L, V, I, F, A, Y;
Z₄ K, R, T, S; and
Z₅ is V, I, L or C.
9. The polynucleotide according to any one of the preceding items, wherein the second polypeptide part is comprising or consisting of an amino acid sequence of the general formula (II):

(SEQ ID NO: X)
Z₁Z₂Z₃Z₄Z₅;

wherein:
Z₁ is H, V, I, A;
Z₂ is W, F, S;
Z₃ is L, V, I;
Z₄ K, R, S; and
Z₅ is V or C.
10. The polynucleotide according to any one of the preceding items, wherein the second polypeptide part is comprising or consisting of an amino acid sequence of the general formula (II):

(SEQ ID NO: X)
Z₁Z₂Z₃Z₄Z₅;

wherein:
Z₁ is H, V, A;
Z₂ is W or S;
Z₃ is L, V, I;
Z₄ K or R; and
Z₅ is V.
11. The polynucleotide according to any one of the preceding items, wherein the second polypeptide part is selected from the group consisting of HWLKV, IETDV, RRTTPV, and YKQTSV.
12. The polynucleotide according to any one of the preceding items, wherein the first polypeptide part is positioned N-terminal to the second polypeptide part.
13. The polynucleotide according to any one of the preceding items, wherein the optional linker is a peptide linker, such as a glycine serine (GS) linker.
14. The polynucleotide according to any one of the preceding items, wherein the optional linker is a glycine serine linker selected from the group consisting of GGS (gLinker2), GGGS (gLinker3), GGGGS (glinker4 SEQ ID NO: 9), GGGGSG (gLinker5), GGGGSGG (gLinker6).
15. The polynucleotide according to any one of the preceding items, wherein the optional linker is GGGGS (glinker4 SEQ ID NO: 9).
16. The polynucleotide according to any one of the preceding items, wherein the polypeptide linker comprises 1 to 12 repeats of the GGGGS moieties (N=1-12).
17. The polynucleotide according to any one of the preceding items, wherein the polypeptide further comprises a cell penetrating peptide (CPP).
18. The polynucleotide according to any one of the preceding items, wherein the CPP is connected to the polypeptide via a linker, such as a polypeptide linker, such as a glycine serine linker.
19. The polynucleotide according to any one of the preceding items, wherein the CPP is positioned N-terminal to the first and the second polypeptide parts.
20. The polynucleotide according to any one of the preceding items, wherein the CPP is selected from the group consisting of TAT, retroinverso-D-TAT, polyarginine, TP10, MAP and PNT.
21. The polynucleotide according to any one of the preceding items, wherein the first polypeptide part is selected from the group consisting of GCN4p1, GCN4p1(7P14P), Atg16, MDV1 and SSO10a,
22. The polynucleotide according to any one of the preceding items, wherein the first polypeptide part is selected from the group consisting of GCN4p1, GCN4p1(7P14P), and SSO10a.
23. The polynucleotide according to any one of the preceding items, wherein the first polypeptide part is GCN4p1 (SEQ ID NO: 8).
24. The polynucleotide according to any one of the preceding items, wherein the first polypeptide part has an amino acid sequence of the general formula (I):

(formula (I), SEQ ID NO: 12)
L-[X]6-L-[X]6-L-[X]6-L,

wherein X is individually selected from any proteinogenic or non-proteinogenic amino acid residue.
25. The polynucleotide according to any one of the preceding items, wherein the first polynucleotide part is capable of forming a leucine zipper.
26. The polynucleotide according to any one of the preceding items, wherein said polynucleotide comprises a sequence encoding upon expression:
   a) a first polypeptide comprising or consisting of an amino acid sequence of the general formula (I): L-[X]₆-L-[X]₆-L-[X]₆-L (SEQ ID NO:12), wherein X is individually selected from any proteinogenic or non-proteinogenic amino acid residue; and
   b) a second polypeptide comprising or consisting of an amino acid sequence of the general formula (II):

(SEQ ID NO: 11)
Z₁Z₂Z₃Z₄Z₅;

wherein:
Z₁ is a proteogenic or non-proteogenic amino acid, preferably H, L, V, I, A; or is absent;
Z₂ is a proteogenic or non-proteogenic amino acid, preferably W, F, T, S;
or is absent;
Z₃ is a proteogenic or non-proteogenic amino acid, preferably L, V, I, F; A, Y;
Z₄ is a proteogenic or non-proteogenic amino acid, preferably K, R, T, S; and
Z₅ is V, I, L or C.
27. The polynucleotide according to any of the preceding items, wherein the first polypeptide part further comprises any proteinogenic or non-proteinogenic amino acid [X], conjugated to the N- and/or C-terminus of the sequence of formula (I).
28. The polynucleotide according to any of the preceding items, wherein the first polypeptide further comprises a tag.
29. The polynucleotide according to any one of the preceding items, wherein the tag is conjugated to the N-terminus of the polypeptide.
30. The polynucleotide according to any one of the preceding items, wherein the tag is an HA-tag.
31. The polynucleotide according to any one of the preceding items, wherein the tag is an c-Myc or His-tag.
32. The polynucleotide according to any one of the preceding items, wherein the tag is a Biotin tag.
33. The polynucleotide according to any one of the preceding items, wherein the biotin tag is operably conjugated to the polypeptide by a 6-aminohexanoic acid (Ahx) linker.
34. The polynucleotide according to any of the preceding items, wherein the second polypeptide binds to a PDZ domain.
35. The polynucleotide according to any of the preceding items, wherein the second polypeptide is capable of inhibiting a protein-protein interaction with the PDZ domain, such as the interaction between AMPAR and PICK1, between cytosolic kinases and PICK1, between synaptic scaffold proteins and PICK1, between membrane embedded proteins and PICK1, between NMDAR and PSD-95, between membrane embedded proteins and PSD-95, or between synaptic scaffold proteins and PSD-95.
36. The polynucleotide according to any of the preceding items, wherein the second polypeptide inhibits the PDZ domain, such as inhibits PICK1, PSD-95, Shank1, Shank2, Shank3, nNOS, syntenin, GRIP, MAGI1, MAGI2, MAGI3, PSD-93, DLG1, ZO-1, Frizzled, PAR3, or PARE, Mint1, or CASK.
37. The polynucleotide according to any of the preceding items, wherein polypeptide has a Ki for the PDZ domain below 25 μM, such as below 10 μM, such as below 5 μM, such as below 1 μM, such as below 800 nM, such as below 600 nM, such as below 400 nM, such as below 300 nM, such as below 200 nM, such as below 160 nM, such as below 150 nM, such as below 140 nM, such as below 130 nM, such as below 120 nM, such as below 110 nM, such as below 100 nM, such as below 90 nM, such as below 80 nM, such as below 70 nM.
38. The polynucleotide according to any one of the preceding items further comprising a promoter sequence.
39. The polynucleotide according to any of the preceding items, wherein said promoter is a human Synapsin1 promoter.
40. The polynucleotide according to any of the preceding items, wherein said promoter is a constitutive promoter.
41. The polynucleotide according to any of the preceding items, wherein said constitutively active promoter is selected from the group consisting of CAG, CBA, CMV, human UbiC, RSV, EF-1alpha, NSE, SV40, Mt1.
42. The polynucleotide according to any of the preceding items, wherein said promoter is an inducible promoter.
43. The polynucleotide according to any of the preceding items, wherein said inducible promoter is selected from the group consisting of Tet-On, Tet-Off, Mo-MLV-LTR, Mx1, progesterone, RU486 and Rapamycin-inducible promoter.
44. The polynucleotide according to any of the preceding items, wherein said promoter is an activity-dependent promoter.
45. The polynucleotide according to any of the preceding items, wherein said activity-dependent promoter is selected from the group consisting of cFos, Arc, Npas4, Egr1 promoters.
46. An expression vector comprising the polynucleotide according to any of the preceding items.
47. The expression vector according to any of the preceding items, wherein the vector is selected from the group consisting of RNA based vectors, DNA based vectors, lipid based vectors, polymer based vectors and colloidal gold particles.
48. The expression vector according to any of the preceding items, wherein the vector is a viral vector.
49. The expression vector according to any of the preceding items, wherein the viral vector is a virally derived DNA vector or a virally derived RNA vector.
50. The expression vector according to any of the preceding items, wherein the vector is selected from the group consisting of adenoviruses, recombinant adeno-associated viruses (rAAV), retroviruses, lentiviruses, adeno-associated viruses, herpesviruses, vaccinia viruses, foamy viruses, cytomegaloviruses, Semliki forest virus, poxviruses, RNA virus vector and DNA virus vector.
51. The expression vector according to any of the preceding items, wherein the expression vector is an adeno associated vector (AAV).
52. The expression vector according to any of the preceding items, wherein the adeno associated vector (AAV) is an AAV1 vector, an AAV2 vector, an AAVS, an AAV8 or an AAV9 vector.
53. The expression vector according to any of the preceding items, wherein the capsid of the AAV1 vector is packaged in an AAV capsid other than an AAV1 capsid.
54. The expression vector according to any of the preceding items, wherein the capsid of the AAV2 vector is packaged in an AAV capsid other than an AAV2 capsid.
55. The expression vector according to any of the preceding items, wherein the capsid of the AAV8 vector is packaged in an AAV capsid other than an AAV8 capsid.
56. The expression vector according to any of the preceding items, wherein the capsid of the AAV9 vector is packaged in an AAV capsid other than an AAV9 capsid.
57. The expression vector according to any of the preceding items, wherein said vector is comprising a sequence encoding upon expression the polypeptide according to any one of the preceding items.
58. The expression vector according to any of the preceding items, wherein said vector is comprising a sequence encoding upon expression a polypeptide comprises or consists of the amino acid sequence of GCN4-GS4-C5 (SEQ ID NO: 6), GCN4(7P14P)-GS4-C5 (SEQ ID NO: 28), GCN4-GS4-IETDV (SEQ ID NO: 30), GCN4(7P14P)-GS4-IETDV (SEQ ID NO: 31), GCN4-GS4-RRTTPV (SEQ ID NO: 33), GCN4(7P14P)-GS4-RRTTPV (SEQ ID NO: 34), GCN4-GS4-YKQTSV (SEQ ID NO: 36), GCN4(7P14P)-GS4-YKQTSV (SEQ ID NO: 37), and SSO10A-GS4-C5 (SEQ ID NO: 46).
59. The expression vector according to any of the preceding items, wherein said vector is functional in mammalian cells.
60. The expression vector according to any of the preceding items, wherein the mammalian cell is a neural cell.
61. The expression vector according to any of the preceding items, wherein the neural cell is a neuron.
62. A polypeptide encoded by the polynucleotide, or the expression vector according to any one of the preceding items.
63. A host cell comprising the polynucleotide, the expression vector or polypeptide according to any one of the preceding items.
64. A composition comprising the polynucleotide, the expression vector or polypeptide according to any one of the preceding items.
65. The composition according to any of the preceding items, wherein the composition is a pharmaceutical composition.
66. The polynucleotide, the expression vector, the polypeptide, the cell, and/or the composition according to any one of the preceding items, for use as a medicament.
67. The polynucleotide, the expression vector, the polypeptide, the cell, and/or the composition according to any one of the preceding items for use in the prophylaxis and/or treatment of a disease and/or disorder associated with maladaptive plasticity and/or transmission.
68. The polynucleotide, the expression vector, the polypeptide, the cell, and/or the composition for use according to any of the preceding items, wherein the disease and/or disorder associated with maladaptive plasticity and/or transmission is selected from the group consisting of neuropathic pain, drug addiction, ischemia, Alzheimer's disease, Parkinson's disease, amyotrophic lateral sclerosis, hearing disorders (e.g. tinnitus), migraine, and epilepsy.
69. The polynucleotide, the expression vector, the polypeptide, the cell, and/or the composition according to any of the preceding items, for use in the prophylaxis and/or treatment of pain in a subject.
70. The polynucleotide, the expression vector, the polypeptide, the cell, and/or the composition for use according to any of the preceding items, wherein the pain is inflammatory pain or neuropathic pain.
71. The polynucleotide, the expression vector, the polypeptide, the cell, and/or the composition for use according to any of the preceding items, wherein the drug addiction is cocaine addiction.
72. The polynucleotide, the expression vector, the polypeptide, the cell, and/or the composition for use according to any of the preceding items, wherein the drug addiction is opioid addiction.
73. The polynucleotide, the expression vector, the polypeptide, the cell, and/or the composition for use according to any of the preceding items, wherein the opioid addiction is morphine addiction.
74. The polynucleotide, the expression vector, the polypeptide, the cell, and/or the composition for use according to any of the preceding items, wherein the disease and/or disorder associated with maladaptive plasticity and/or transmission is Alzheimer's disease.
75. A method of treatment or prevention of a disease and/or disorder associated with maladaptive plasticity and/or transmission in a subject in need thereof, comprising administering a therapeutically effective amount of the polynucleotide, the expression vector, the polypeptide, the cell, and/or the composition according to any one of the preceding items.
76. Use of the polynucleotide, the expression vector, the polypeptide, the cell, and/or the composition according to any one of the preceding items, for the manufacture of a medicament for the prophylaxis and/or treatment of a disease and/or disorder associated with maladaptive plasticity and/or transmission.

[symbol]

### EXAMPLES

The following examples demonstrate that polypeptides comprising a first polypeptide part capable of forming a dimer and a second polypeptide comprising a PDZ binding domain (PBM) can obtain high affinity binding to PDZ domains. Upon expression in tissue, including spinal cord/dorsal root ganglion (DRG), the polypeptide can fully alleviate symptoms of pain and upon expression in the hippocampus improve the cognitive ability in a model of Alzheimer's disease.

### Example 1

### Materials and Methods

### Design of a High Affinity Inhibitor of PICK1

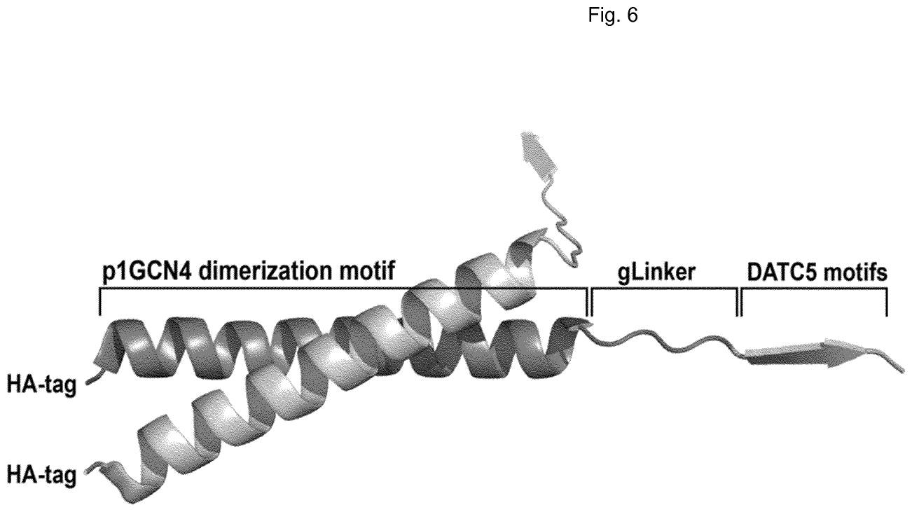

It was hypothesized that a recombinant polypeptide which was capable of forming a dimeric structure would further increase the affinity towards PICK1 and PDZ domains. Hence, a synthetic encoding DNA sequence that upon delivery by a vector to a living cell, would first be transcribed and translated into a single-stranded polypeptide and then potentially assembled into a dimeric peptide, was constructed. Inspired from the structural features of the leucine zipper of the yeast bZIP transcription factor GCN4 (Gonzarlez et al 1996, Nat. Struct. Biol.; Lee et al 2014 Nat. Commun.; Oshaben et al 2012 Biochemistry; O'Shea et al 1991 Science), a recombinant polypeptide that form a stable homodimeric structure tethered with two DATCS motifs was genetically engineered. A DNA vector encoding the protein sequence of the leucine zipper (coiled-coil domain) of GCN4, called GCN4p1 or p1GCN4, devoid of the DNA binding domain of GCN4 was synthesized. This GCN4p1 sequence encodes a short structure that form a homodimeric structure. In addition, a GS (glycine serine) linker was attached to the N-terminus upon which a HA-tag (human influenza hemagglutinin molecule corresponding to amino acid 98-106; YPYDVPDYA) was attached to the N-terminus of GS, whereas a glycine rich linker (GS region) terminating with the DATCS motif was attached to the C-terminus of p1GCN4. This DNA sequence had a 5′start codon (ATG) and was terminated with a 3′stop codon (TAA). The resulting recombinant polypeptide was termed HA-GCN4-GS4-C5. A model of the self-assemble propensity of the dimeric structure of HA-GCN4-GS4-C5 polypeptide is graphically shown in FIG. 6.

### 1. Plasmid Design

The DNA region spanning the entire coding sequence of HA-GCN4-GS4-C5 oligopeptide with appropriate 5′ and 3′ restriction sites was ordered as a pre-manufactured circular plasmid, pEX, from Eurofins Genomics. In this example, a GS region containing the amino acid composition: G-G-G-G-S(GS4) was used. The DNA insert was next by traditional “cut and insert” cloning technique inserted into a generic AAV plasmid backbone. This AAV plasmid backbone contained an upstream human Synapsin1 (pan-neuronal) promoter, followed by a multiple cloning site (MCS, containing similar restriction sites as found in the flanking region of the oligopeptide DNA sequence), and terminated by WPRE and Poly A signal. This DNA sequence within the AAV plasmid backbone back was flanked by the 5′- and 3′-ITRs. The HA-GCN4-GS4-C5 oligopeptide sequence was inserted into the MCS via its restrictions sites. Correct insertion and integrity of the pAAV plasmid was finally confirmed by PCR sequencing. pAAV plasmids with HA-GCN4-GS4-V-to-D mutant and HA-GCN4-GS4-7P14P mutant coding sequences were produced similarly as described above. The genetic encoded vector pAAV-hSyn-tdTomato was used as a control.

### 2. Viral Production

All AAV viruses were generated in-house using a FuGene6 mediated triple plasmid co-transfection method in HEK293FT cells. These procedures have been described earlier (Sørensen et al., 2016, eLife). For the triple transfection, AAV pHelper plasmid, AAV Rep(2)-Cap(8) plasmid and our AAV plasmid vectors were used. Three days after transfection, cells were harvested and virus was purified using an adapted Iodixanol gradient purification protocol. Genomic AAV titer was determined by a PicoGreen-based method. Before use, all viruses were carefully examined in Western Blots for purification, and, if needed, diluted in DPBS for optimized titer. The final AAV vectors were:

### 1. AAV8-hSyn-HA-GCN4-GS4-C5-WPRE-pA

### 2. AAV8-hSyn-HA-GCN4-GS4-D-V-WPRE-pA

### 3. AAV8-hSyn-HA-GCN4-GS4-7P14P-WPRE-pA

### 3. Synthetic Peptides

The following synthetic peptides were used in the experiments: Synthetic variants of

GCN4-GS4-C5, GCN4-GS4-7P14P, or GCN4-GS4-V-to-D, with an N-terminal Biotin and an Ahx linker.

### Conclusion

This example demonstrates how viral constructs were prepared, and how equivalent synthetic peptides were designed. The procedure was applied to other viral constructs and synthetic peptides of the present disclosure.

### Example 2

### Materials and Methods

### Protein Expression and Purification of PICK1

E. Coli cultures (BL21-DE3-pLysS) transformed with a PICK1 encoding plasmid (pGEX4T2)(Madsen et al., 2005), was inoculated in LB with Ampicillin medium overnight and transferred into Luria-Betani (LB) medium with Ampicillin and grown at 37° C. until OD600=0.6. Protein expression was induced with 10 mM Isopropyl β-D-1-thiogalactopyranoside (IPTG) and grown overnight at 20° C. Bacteria were harvested and resuspended in lysis buffer containing 50 mM Tris, 125 mM NaCl, 2 mM DTT, 1% TritonX-100, 20 μg/ml DNAse 1 and half a tablet of complete protease inhibitor cocktail pr. 1 L culture. Resuspended pellet was frozen at −80° C. to induce cell lysis. The bacterial suspension was thawed and cleared by centrifugation. The supernatant was collected and incubated with Glutathione-Sepharose 4B beads for 1 hour at 4° C. under gentle rotation. The beads were pelleted at 4000×g for 5 min and supernatant was removed and beads were washed 2 times in 50 mM Tris, 125 mM NaCl, 2 mM DTT and 0.01% Triton-X100 pH 7.4. Washed beads were transferred to an empty gravity column with a filter bed. Bead solution was incubated with thrombin protease (Millipore) overnight at 4° C. under gentle rotation. PICK1 was eluted on ice and absorption at 280-nm was measured on a NanoDrop2000, and protein concentration was measured using lambert beers law (A=cc1), cA280PICK1=32320 (cm*mol/L)⁻¹.

### Size Exclusion Chromatography

GCN4-GS4-C5 (10 μM), GCN4-GS4-7P14P (10 μM), or GCN4-GS4-V-to-D (10 μM) was incubated in absence or presence of PICK1 (40 μM) in 50 mM Tris, 125 mM NaCl, 2 mM DTT, 0.01% TX100, pH 7.4 and sample oligomeric composition was validated using SEC (Superdex200 Increase 10/300) measuring the absorbance at 280 nm.

### Results

GCN4-GS4-C5 Effectively Dimerizes and Shifts PICK1 into a Tetrameric Configuration

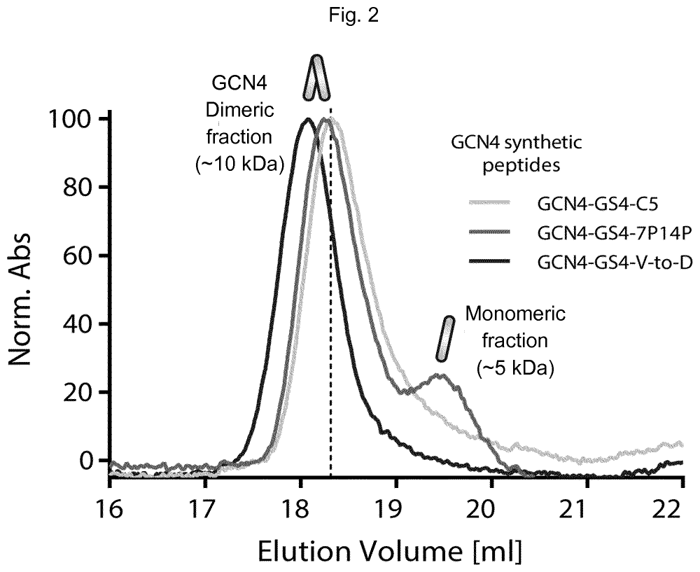

Using size exclusion chromatography it was demonstrated that the synthetic GCN4-GS4-C5 peptide eluted as a single peak at ˜18.3 ml. GCN4-GS4-V-to-D likewise eluted exclusively in peak at ˜18 ml indicating intact dimer formation. GCN4-GS4-7P14P, which has two prolines inserted into the GCN4 sequence to compromise dimerization (Leder et al 1995 Biochemistry), on the other hand, is partly shifted to a peak at 19.8 ml indicating partially disruption of the dimerization (FIG. 2). Hence, GCN4-GS4-7P14P was found as a mixture of dimers and monomers at the conditions of the experiment.

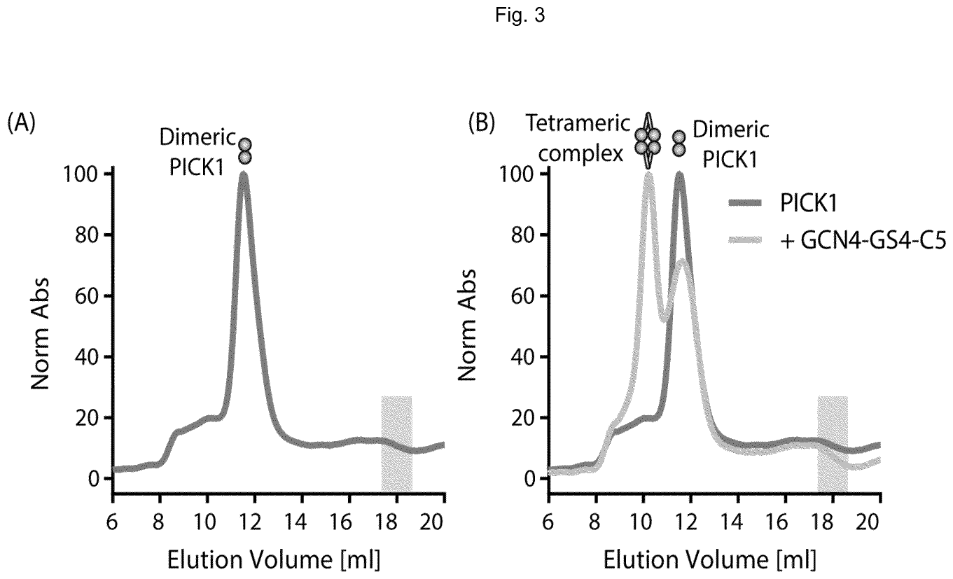

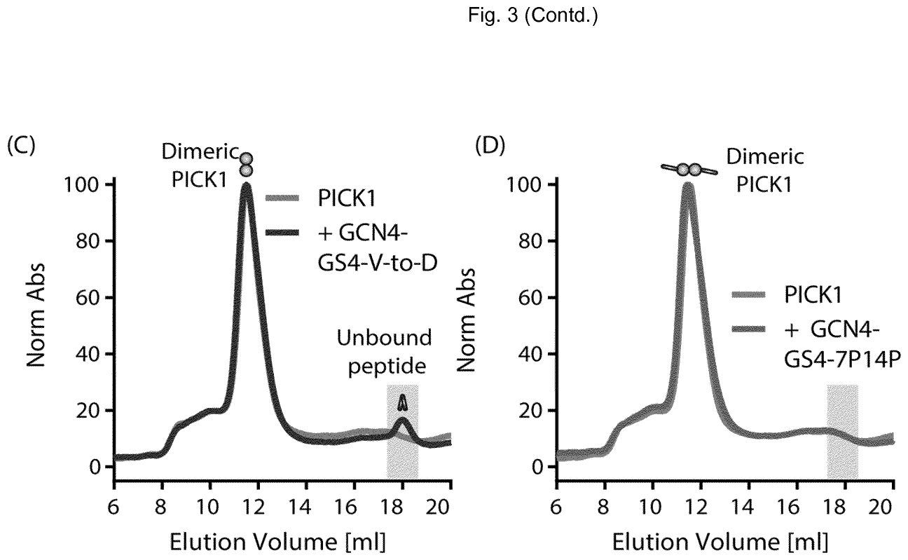

Upon incubation of PICK1 (40 μM) with the GCN4-GS4-C5 (10 μM), PICK1 shifted partly from an elution at 11.7 ml (FIG. 3A), which represents the dimeric form of the protein, to an elution at ˜10 ml indication formation of the tetrameric configuration of PICK1 critical for obtaining high affinity binding (FIG. 3B). GCN4-GS4-V-to-D, designed to have the PDZ interacting sequence disrupted, did not cause the shift in PICK1 elution (11.7 ml) (FIG. 3C), however, an unbound fraction of the peptide eluted at ˜18 ml, in agreement with FIG. 2 and disrupted binding to PICK1. Finally, PICK1 (40 μM) incubated with GCN4-GS4-7P14P (10 μM) also eluted at 11.7 ml (FIG. 3D), indicating that this peptide construct was not capable of inducing tetramer formation of PICK1.

### Example 3

### Materials and Methods

### Fluorescence Polarization In Vitro Binding to PICK1

Fluorescence polarization was carried out in saturation mode and competition mode. In brief, saturation experiments were carried out using an increasing amount of protein incubated with a fixed concentration of fluorescent tracer 5FAM-hDAT-05 or PEG4 linked dimeric hDATC5 20 nM. Competition was performed at a fix concentration of protein and tracer, against an increasing concentration of unlabeled peptide. The fluorescence polarization was measured after 17-20 hours of incubation at 4° C. on an Omega POLAR star plate reader using excitation filter at 488-nm and long pass emission filter at 535-nm.

### Results

GCN4-GS4-C5 Binds to PICK1 with High Affinity Through the PDZ Domain

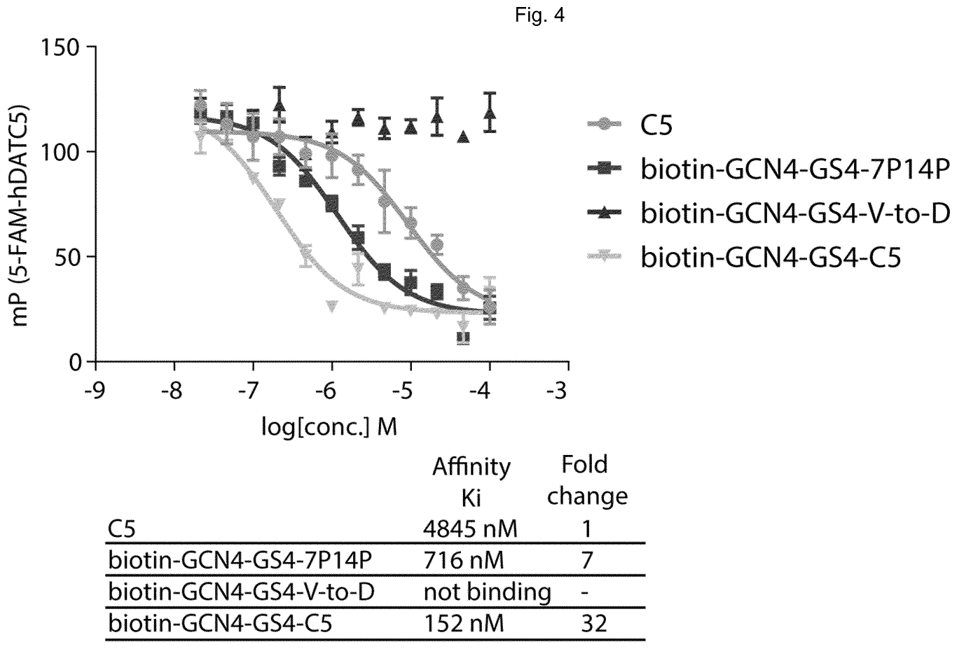

Fluorescent polarization experiments were performed to determine binding affinity for PICK1. Competition experiments using the DAT C5 peptide as fluorescent tracer demonstrated a 32-fold increase in affinity of GCN4-GS4-C5 over the DAT C5 peptide to an apparent affinity of 151 nM. GCN4-GS4-7P14P had a 5-fold reduced affinity compared to GCN4-GS4-C5. Finally, GCN4-GS4-V-to-D, designed to have the PDZ interacting sequence disrupted, showed no binding to PICK1 (FIG. 4).

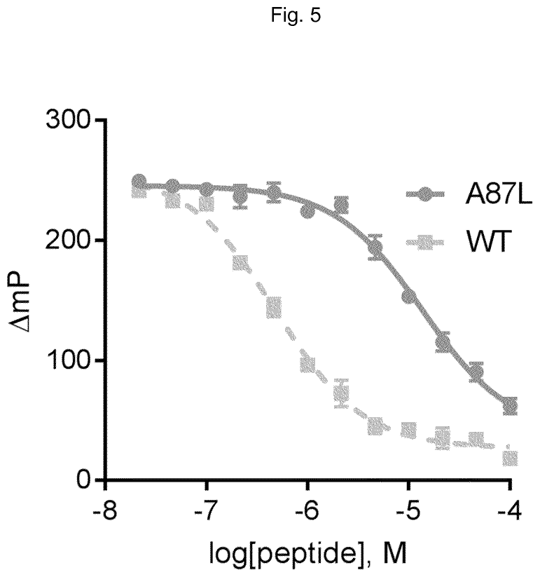

To confirm that the interaction of GCN4-GS4-C5 was mediated by the PICK1 PDZ domain, the PICK1 A87L, which has a mutation in the PDZ domain peptide binding groove, was used. Indeed, GCN4-GS4-C5 showed 18-fold lower affinity for PICK1 A87L than for PICK WT, demonstrating strong dependence on the PDZ domain for binding (FIG. 5).

### Example 4

### Material and Methods

### Immunohistochemistry of Spinal Horn Tissue

### 1. Surgery and Virus Injection

Mice were initially anaesthetized with 4% isoflurane and kept under constant anaesthesia (1.8-2% isoflurane) during surgery and virus injection. The hair on the back was shaved off and 70% ethanol was sprayed on. The back was opened by a small skin incision at the level of vertebrae L2 (i.e. vertebrae 5 as counted from the rear of the spinal cord) to visualize the spinal cord. The dorsal artery was visible and lined the separation of the lateral sides. A glass pulled pipette, filled with virus and attached to a Stoelting injection pump, was inserted halfway through the spinal cord (approx. 1 mm) on both sides of the dorsal artery to perform a bilateral injection. 0.5 μL virus (AAV8-hSyn-HA-GCN4-GS4-C5-WPRE-pA) was injected on each side at a rate of 0.1 μL/min via the automatic pump. Once injected, the needle was kept in place for 5 min to avoid leakage through the injection site. The skin of the back was glued together to close the incision. The mice recovered fully following surgery. All described procedures were approved by the local ethical committee on the use of experimental animals.

### 2. Perfusion and Spinal Cord Dissection

Three weeks following virus injection, mice were completely anesthetized with pentobarbital (i.p. 50 mg/kg). Mice were perfused first with ice-cold PBS then with 4% PFA, and the vertebral column was separated in a tissue block, which was left in 4% PFA for 1 h. The spinal cord was further dissected by cutting the sides of the vertebrae along the rostro-caudal axis, and the bone and other tissues were torn off. The spinal cord was gently lifted to avoid damage when cutting and kept hydrated during dissection. After dissecting out the spinal cord, it was left in 4% PFA for another 1 h in horizontal position on ice. Afterwards, the tissue was transferred to 30% sucrose in Milli-Q H₂O until saturated (i.e. non-floating). The tissue was stored in antifreeze (15 μM NaH₂PO₄, 38 μM Na₂HPO₄, 30% ethylenglycol, 30% glycerol, 40% MQ H₂O) at −20 degrees until use. Before embedding, the tissue was placed in 30% sucrose as described above until saturated. The lumbar and thoracic parts of the spinal cord was separated and snap frozen in moulds with Tissue-Tek O.C.T Compound by adding dry ice to isopentane. Spinal cords were cut on cryostat with a 30 μm thickness and transferred to cold PBS-filled 24-well plates. Several washes with PBS were performed to remove O.C.T.

### 3. Immunohistochemistry

Free-floating sections were blocked with 5% donkey serum in PBST (PBS+Triton X-100 0.1%) for 1 h at RT before incubated with primary antibody against HA (human influenza hemagglutinin molecule corresponding to amino acid 98-106; YPYDVPDYA) raised in rabbit at a concentration of 1:100 in PBST with 5% donkey serum at 4 degrees for 5 days. Three washes with PBS were performed for 1 h at RT. Then the sections were incubated with secondary antibody (Donkey anti-Rabbit IgG; Alexa Flour 488) at a concentration of 1:500 in PBS with 5% donkey serum for 1 h at RT in darkness. Three washes in PBS for 2 h were performed. The sections were mounted on Superfrost Menzel-Glaser Thermo Scientific microscope glasses and dried in fumehood for 2 h. Coverslips were mounted with ProLong™ Gold Antifade Mountant with DAPI.

### 4. Imaging

Microscopy was performed on a Zeiss Axiovert 100 equipped with epifluorescence. DAPI was visualized using a 10× objective (exposure time of 1 sec, lx gain), while Alexa Flour 488 to visualize HA-staining was performed with a 10× or 20× objective (exposure time of 4 sec, 3× gain).

### Results

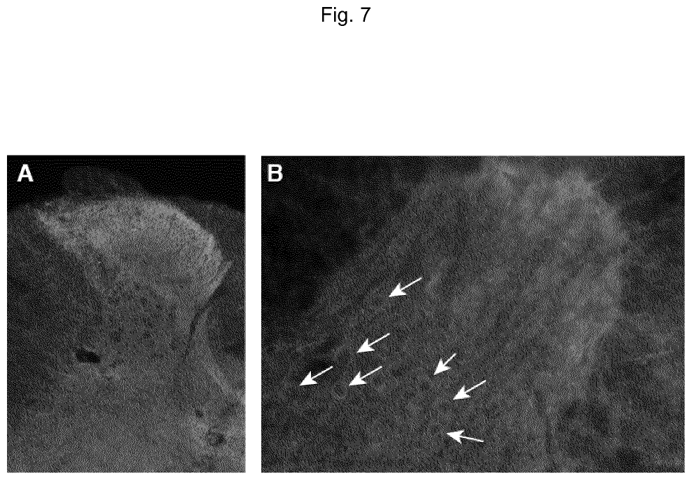

Intrathecal AAV8-hSyn-HA-GCN4-GS4-C5-WPRE-pA administered conferred expression towards the grey matter of the spinal cord as visualized for positive HA-stain (seen as brighter area) at the vertebra L1-L2 level (FIG. 7A; 10× objectives). This expression appears confined towards neurons within the spinal horn (FIG. 7B; marked by white arrows, 20× objective), as also predicted by the use of the human Synapsin (hSyn) promoter.

### Example 5

### Materials and Methods

### Mouse Spared Nerve Injury (SNI) Model

### 1. Animals

For the study, C57BL/6 mice were used. After delivery, mice habituated for approximately 1 week before start of experiments. Animals had access to food and water ad libitum. A 12 h light/12 h dark cycle was used, and the experimental procedures were performed during the light cycle. All described procedures were approved by the local ethical committee on the use of experimental animals.

### 2. Virus Preparation and Administration

Virus was administered intrathecally in a volume of 5 μl to anesthetized mice with 10 μl Hamilton syringe and 30G needle in the intervertebral space between L5/L6 (in order to minimize the possibility of spinal damage). Prior to injection, the virus was diluted 1:5 in DPBS. After dilution, the viral titers of AAV8-hSyn-HA-GCN4-GS4-C5-WPRE-pA and AAV8-hSyn-tdTomato were 2.74E+12 vg/mL and 2.12E+12 vg/mL, respectively. With intrathecally injections, the correct positioning of the needle was assured by a typical flick of the tail.

### 3. Surgical Procedure

Spared nerve injury model (SNI) was performed according to methods described in Richner et al., 2011. Briefly, under isoflurane (2%) anesthesia, the skin on the left lateral surface of the thigh was incised and the biceps femoris muscle was divided and spread lengthwise to expose the three branches of the sciatic nerve. After exposing the three branches (common peroneal, tibial and sural) in anesthetized mice, the common peroneal and tibial branches were carefully segregated from surrounding tissues, tightly ligated and axotomized −2 mm of the distal nerve stump, while the sural branch was left intact. Animals were monitored daily for signs of stress or discomfort but in all cases recovered uneventfully.

### 4. Behavioral Assessments

The development and level of mechanical threshold was determined both before and after the SNI procedure by using Von Frey filaments ranging from 0.02 to 1.4 g. Both the ipsilateral (injured) and contralateral (intact) hind paw was used for the testing. In the current experiments filaments in ascending order were applied to the lateral part of the hind paws. Mice were placed in a red colored plastic cylinders placed on a wire mesh in order to let them habituate for 15 min in cylinders prior to testing. Each Von Frey hair was applied five times over a total period of 30 seconds (approximately 2 seconds per stimulus) and the mouse's reaction was assessed after each application; the threshold for a positive test was set at 3 trials, which evoked responses out of a maximum of 5 trials. A positive pain reaction is defined as sudden paw withdrawal, flinching and/or paw licking induced by the filament. Furthermore, a positive response in three or more out of five repetitive stimuli is defined as the pain threshold. Forces of the instrument are measured with units “g” and represent the gram-forces. The paw withdrawal threshold (PWT) was estimated by using the following formula: PWT=(Number of response failures)/(Total number of trials)×((filament A+1 gr)−(filament A gr))+filament A gr.

### 5. Overview of Experimental Timeline

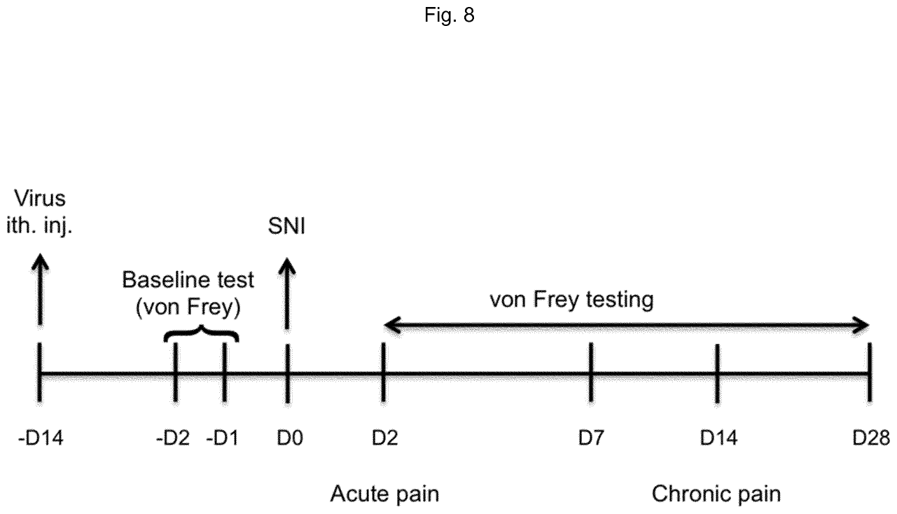

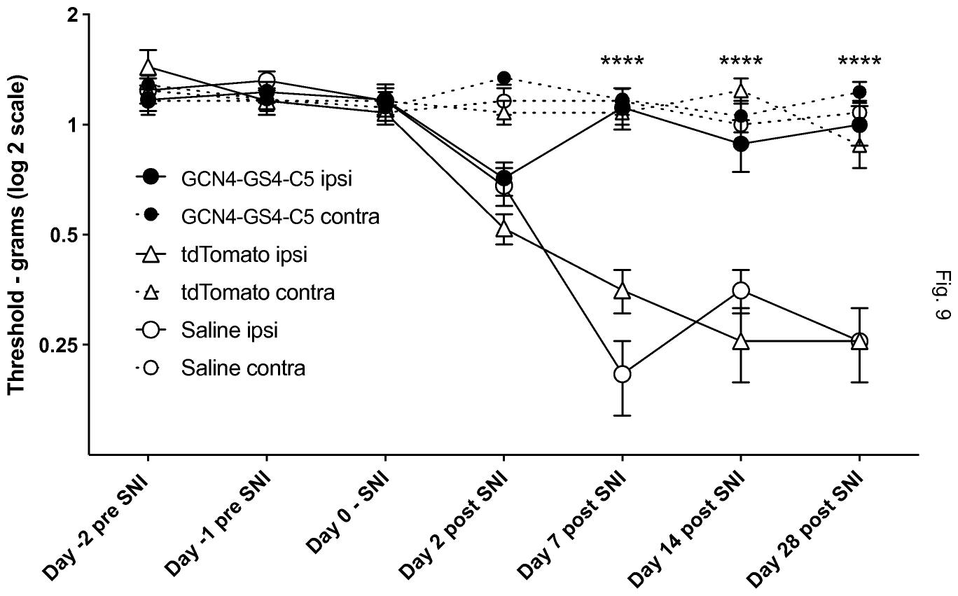

The SNI behavioral experiment was performed during a 6 week period as outlined in FIG. 8.

### 6. Immunohistochemistry of Spinal Cord Tissue.

After end experiments, all mice were deeply euthanized with isofluorane and then decapitated with scissors. The skin of the back was cut open and 70% ethanol sprayed on. The vertebral column was separated from the rest of the tissue by cutting on each side of it in a rostro-caudal direction until the hips of the mouse were reached. A horizontal cut across the lower back was performed to free the tissue block. A pipette tip was inserted into the spinal cavity in the rostral end (tail end) and through steady pressure; the spinal cord was pressed out with cold PBS into a petri-dish with cold PBS. The spinal cords were next transferred to 4% PFA on ice in for 6 hours, before being transferred to 30% sucrose at 4 degrees until saturated (non-floating). The lumbar and thoracic parts of the spinal cord was separated and snap frozen in moulds with Tissue-Tek O.C.T Compound by adding dry ice to isopentane, and then stored at −80 degrees. Spinal cords were cut on a cryostat at 30 μm thickness and transferred to cold PBS-filled 24-well plates. Several washes with PBS were performed to remove O.C.T. For staining, free-floating sections were blocked with 5% donkey serum in PBST (PBS+Triton X-100 0.1%) for 1 h at RT before incubation with primary antibody at a concentration of 1:100 in PBST with 5% donkey serum at 4 degrees for 5 days. Three washes with PBS were performed for 1 h at RT. Then the sections were incubated with secondary antibody (Donkey anti-Rabbit IgG; Alexa Flour 488) at a concentration of 1:500 in PBS with 5% donkey serum for 1 h at RT in darkness. Three washes in PBS for 2 h were performed. The sections were mounted on Superfrost Menzel-Glaser Thermo Scientific microscope glasses and dried in fumehood for 2 h. Coverslips were mounted with ProLong™ Gold Antifade Mountant with DAPI. Images were obtained using AxioScan.Z1 microscope.

### 7. Transgene Expression in Mice Exposed to SNI.

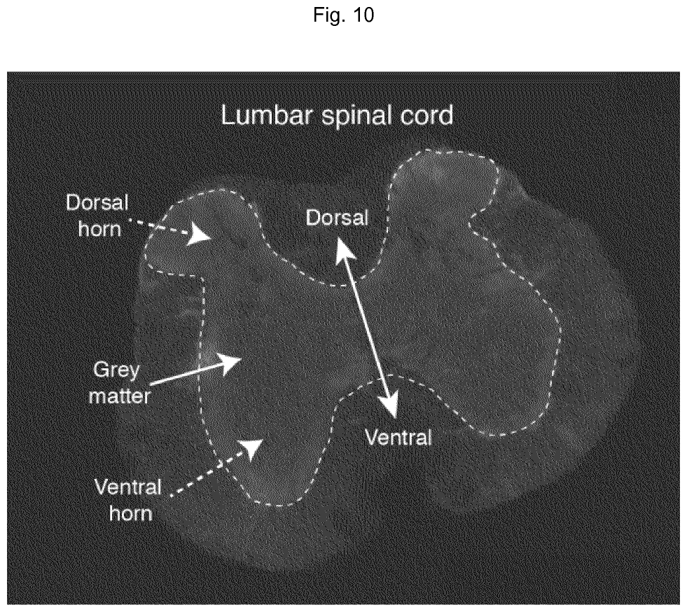

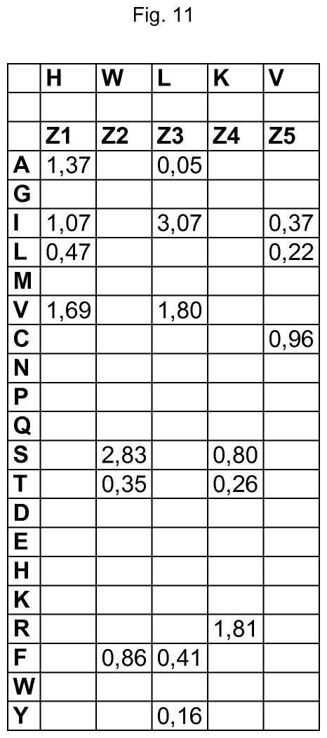

Intrathecal AAV8-hSyn-HA-p1GCN4-gLinker4-DATC5 administered confers expression towards the grey matter throughout the spinal cord as visualized for positive HA-immunoreactivity (bright white color) at the lumbar vertebra level (FIG. 10).

### Results

AAV8-hSyn-HA-GCN4-GS4-C5-WPRE-pA treatment completely and persistently alleviates mechanical hyperalgesia in the mouse SNI model (FIG. 8-9). Animals were injected intrathecally with saline, AAV8-hSyn-tdTomato (control vector) (tdTomato) or AAV8-hSyn-HA-GCN4-GS4-C5-WPRE-pA (GCN4-GS4-C5). Von Frey test shows complete pain relief for AAV8-hSyn-HA-GCN4-GS4-C5-WPRE-pA treated mice at day 7, day 14 and day 28 post SNI, as compared to its own intact paw (contra), and as compared to control animals (Saline ipsi and tdTomato ispi). Data was analyzed using 2-way ANOVA with Tukey's post-hoc test, F(30, 196)=6,511, p<0.0001.

### Example 6

### Variants of the PDZ Binding Peptide (DA T-C5, SEQ ID NO: 10)

To test the stringency of the PICK1 PDZ binding motif in the DAT C5 sequence (i.e. position Z₁-Z₅) and to indicate putatively peptides with better affinity, we performed fluorescence polarization binding of C5 peptides to purified PICK1 with each residue in the HWLKV sequence substituted to the indicated amino acids.

### Materials and Methods

PICK1 was expressed and purified as described in example 2.

Fluorescence polarization: Fluorescence polarization was carried out in competition mode at a fixed concentration of protein and tracer (5FAM-DATC5, 20 nM), against an increasing concentration of unlabelled C5 peptide with point substitutions as indicated. The plate was incubated 20 min on ice in a black half-area Corning Black non-binding surface 96-well plate and the fluorescence polarization was measured directly on an Omega POLARstar plate reader using excitation filter at 488-nm and long pass emission filter at 535-nm. The data was plotted using GraphPad Prism 6.0, and fitted to the One site competition, to extract Kd values, which were All correlated to the WT C5 affinity, which was finally plotted.

### Results

Substitution in general were well tolerated however with lower tolerance in position Z3 and Z5 in agreement with the important role for these residues in PDZ specificity. Notable increases in affinity were Z1 to V, Z2 to S, Z3 to I and V, and Z4. No substitutions at Z5 increased affinity (FIG. 11).

### Example 7

### Variants of PDZ-Binding Ligands to Target Other PDZ Domains

A study of other C-terminal peptides that can target any PDZ domain containing protein using class I, II and III PDZ domain binding motif (PBM) was conducted to investigate the potency of the dimeric constructs towards other PDZ domains.

Class I PBM is defined by the consensus sequence -T/S-¥-Ψ, Class II is defined by the consensus sequence -Ψ-¥-Ψ and class III is defined by the consensus sequence -D/E-¥-Ψ, where Ψ is defined as hydrophobic amino acids and ¥ is any proteinogenic amino acid. In PSD-95, all PDZ domains favors class I PBM's, but with different specificity for the remaining amino acid positions.

### Materials and Methods

Protein expression and purification of FL-PSD-95: E. Coli cultures (BL21-DE3-pLysS) transformed with a TRX-6×His-hPSD95 1-724 encoding plasmid (pET-MG-3C) (Zeng et al. 2016), was inoculated in Luria-Betani (LB) media supplemented with ampicillin and chloramphenicol overnight and transferred into LB medium supplemented with ampicillin and chloramphenicol and grown at 37° C. until OD600=0.6. Protein expression was induced with 1 mM Isopropyl β-D-1-thiogalactopyranoside (IPTG) and grown for 8-16 hrs at 16-20° C. Bacteria were harvested and frozen at −80° C. Pellet was thawed and resuspended in 50 mM Tris (pH 8.0), 300 mM NaCl, 1 mM TCEP, 20 μg/μl DNAse, 1 tablet of cOmpete Protease inhibitor pr. 1 L culture. Resuspended bacteria were sonicated for 2 minutes to induce lysis and lysaste was cleared by centrifugation at 30.000 g for 20 min. The supernatant was collected and run through to a 5 ml HisTrap HP column and column was washed with 50 mM Tris (pH 8.0), 300 mM NaCl, 10 mM Imidazole, 1 mM TCEP. Bound protein was eluted using a linear gradient from 10-500 mM Imidazole in 50 mM Tris (pH 8.0), 300 mM NaCl, 1 mM TCEP. Protein containing fractions were pooled and purified further using a Superdex 200 μg 1.6/600 Size exclusion column equilibrated in 50 mM Tris (pH 8.0), 300 mM NaCl, 10 mM EDTA, 1 mM TCEP. Protein purity was validated to be at least above 90% using SDS-PAGE, UPLC and LC-MS.

Fluorescence polarization: Fluorescence polarization was carried out in competition mode at a fixed concentration of protein (150 nM) and tracer (5FAM-NPEG4(1ETAV)₂, 5 nM), against an increasing concentration of unlabeled peptide. The plate was incubated 2 hrs on ice in a black half-area Corning Black non-binding surface 96-well plate and the fluorescence polarization was measured directly on a Omega POLARstar plate reader using excitation filter at 488-nm and long pass emission filter at 535-nm. The data was plotted using GraphPad Prism 6.0, and fitted to the One site competition, to extract KI values.

Circular dichroism (CD): Circular dichroism (CD) spectra was recorded using a Jasco J1500 at 25° C. spectrum was recorded from 190-260 nm in 0.1 nm intervals, using a 1 mm cuvette. Indicated peptides were diluted to 8 μM in 50 mM Sodium Phosphate (NaPi) buffer (pH 8), and spectra was collected.

### Results

To establish proof-of-concept of the strategy of the present disclosure, we chose to target the postsynaptic density protein 95 (PSD-95), a major PDZ containing scaffolding protein found in the excitatory postsynaptic density.

### PSD-95 PDZ1-2 Binding PBM:

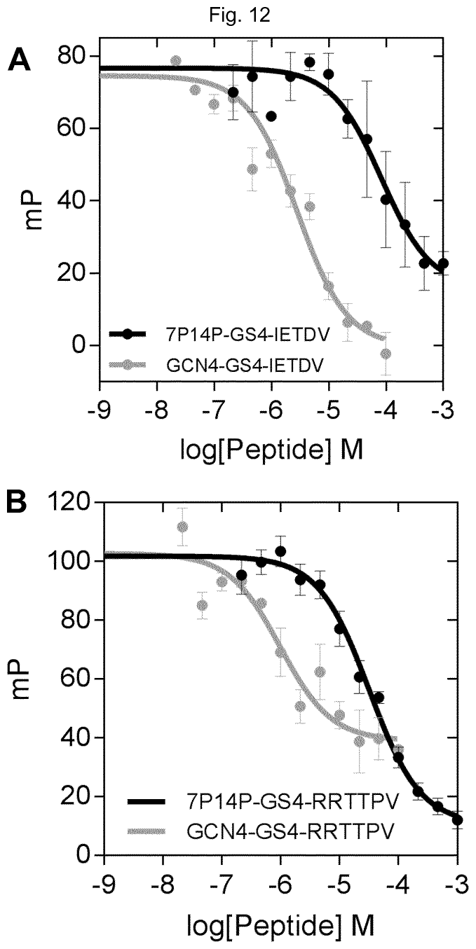

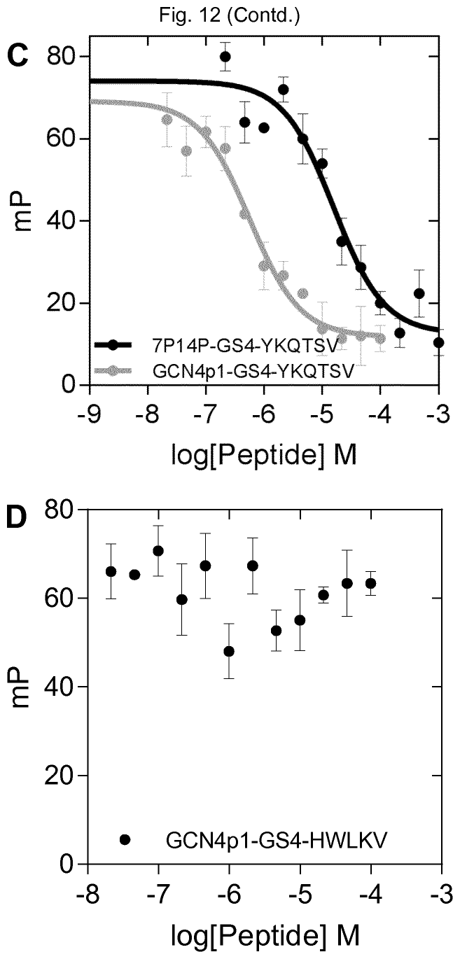

First, we chose a PDZ1-2 specific PBM amino acid sequence, IETDV, previously shown to have high affinity and specificity for PSD-95 PDZ1-2 domain (Bach et al., 2012). For this, we tested a GCN4-GS4-IETDV and 7P14P-GS4-IETDV variant. We found using fluorescent polarization (FP) assay that GCN4p1-GS4-IETDV had a Ki value of 0.7 μM towards PSD-95. The 7P14P-GS4-IETDV variant had a Ki value of 20.8 μM towards PSD-95 (FIG. 12A).

### PSD-95 PDZ1-3 Binding PBM:

We designed a PSD-95 PDZ promiscuous variant (GCN4p1-GS4-RRTTPV), derived from the AMPAR receptor Auxiliary protein TARP-γ2/Stargazin, with the C-terminal sequence RRTTPV, supposedly able to bind all PDZ domains in PSD-95 (Sainlos et al. 2011) and variant 7P14P-GS4-RRTTPV. Using FP, we found that GCN4p1-GS4-RRTTPV had Ki value of 0.25 μM towards PSD-95, whereas the 7P14P-GS4-RRTTPV variant had Ki value of 8.0 μM towards PSD-95 (FIG. 12B).

### PSD-95 PDZ3 Binding PBM:

We designed a PSD-95 PDZ3 specific variant (GCN4p1-GS4-YKQTSV), derived from the neuronal adaptor protein Cysteine-rich PDZ-binding protein (Cript), with the C-terminal sequence YKQTSV, supposedly with >15-fold specificity towards PDZ3 over PDZ 1 and 2 in PSD-95 (Toto et al. 2016) and variant 7P14P-GS4-RRTTPV. Using FP, we found that GCN4p1-GS4-YKQTSV had a Ki value of 0.14 μM towards PSD-95, whereas 7P14P-GS4-YKQTSV had a Ki value of 3.9 μM towards PSD-95 (FIG. 12C).

### Negative Control Experiment:

A specificity control experiment was performed studying the affinity of the PICK1 binding variant, GCN4p1-GS4-HWLKV towards PSD-95. This peptide was not capable of binding PSD-95 (FIG. 12D).

### Tertiary Structure Analysis by Circular Dichroism:

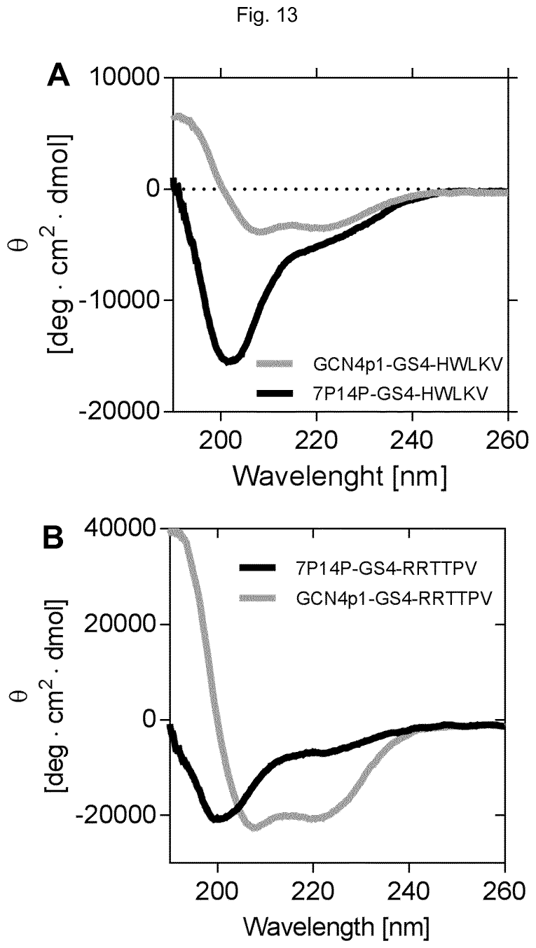

Circular dichroism (CD) measurements showing helical structure of different variants of GCN4-GS4-X binding proteins (FIG. 13A-B) was performed. GCN4-GS4-HWLKV shows typical alpha-helix signature with local minima at 208 and 222 nm, while the variant 7P14P show random coil indicating disruption of the helical structure (FIG. 13A). Similar structural features were observed for GCN4p1-GS4-RRTTPV and 7P14P-GS4-RRTTPV (FIG. 13B).

### Conclusion

These examples provide proof-of-principle that the constructs of the present disclosure provide means for selective targeting of various PDZ domains, with the PBMs selective for the respective PDZ domain with increased affinity.

### Example 8

### Variants of Amphipathic Helix

A study of other polypeptides which are capable of forming a dimer was conducted to investigate the potency of the constructs towards PDZ domains. Examples of other polypeptides capable of forming a dimer include without limitation Atg16, MDV1, and SSO10a.

In this experiment, we tested whether the a4 helix of SSO10a protein can serve as an alternative backbone to achieve high affinity binding toward PICK1. The SSO10a proteins are small DNA-binding proteins expressed by the crenarchaeal model organism Sulfolobus solfataricus (Chen et al. 2004).

In this series of experiment, the following peptides were studied;

SSO10a-GS4-HWLKV
(SEQ ID NO: 46):
GEELLEDIRKFNEMRKNMDQLKEKINSVLSIRQ-GGGGS-HWLKV
GCN4-G54-HWLKV
(SEQ ID NO: 6):
RMKQLEDKVEELLSKNYHLENEVARLKKLV-GGGGS-HWLKV
GCN4-G54-G54
(SEQ ID NO: 38):
RMKQLEDKVEELLSKNYHLENEVARLKKLV-GGGGS-GGGGS

All three peptides furthermore contained an N-terminal Biotin and an Ahx linker.

### Materials and Methods

PICK1 was expressed and purified as described in example 2.

### Fluorescence Polarization:

The competition binding assay was carried out using a fixed concentration of PICK1 (0.15 μM) and fluorescent PEG4 linked dimeric C5 tracer (20 nM) incubated with increasing concentrations of unlabelled peptides using black half-area Corning non-binding surface 96 well plates (Sigma-Aldrich, Ref. no. 3686). The plates were incubated 48 hrs at 4 degrees and the fluorescence polarization was measured on an Omega POLARstar plate (BMG LABTECH) reader using excitation filter at 485 nm and long pass emission filter at 535 nm. The data was plotted in GraphPad Prism 7.0 and fitted to a ‘One site—Fit’ K curve and the apparent affinities (K) for the unlabelled peptides were determined using correction for depletion.

### Fast Protein Liquid Chromatography—FPLC:

FPLC size exclusion chromatography was done using a Akta purifier with a Superdex200 Increase 10/300 column, where 500 μL of 40 μM PICK1 in absence or presence of 20 μM GCN4-GS4-HWLKV, GCN4-GS4-GS4 or SSO10a-GS4-HWLKV respectively. Absorbance profile was measured at 280 nm and plotted against elution volume using Graph Pad Prism.

### Results

### Fluorescent Polarization

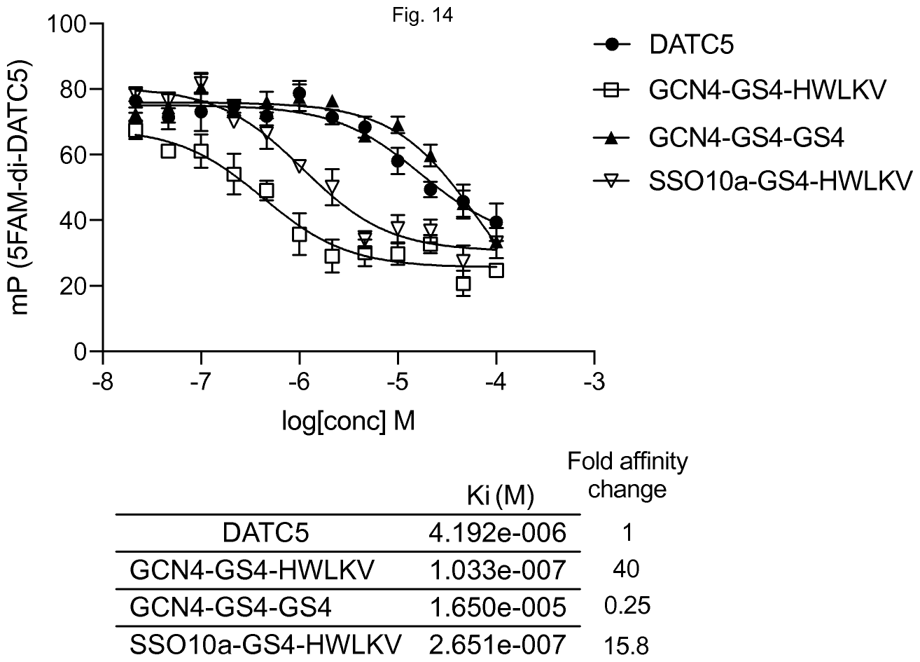

Fluorescent polarization (FP) experiments were performed to determine binding affinity for PICK1. Competition experiment using fluorescent PEG4 linked dimeric C5 tracer demonstrated the highest affinity for GCN4-GS4-HWLKV (SEQ ID NO: 6), approx. a 40-fold shift compared to DATCS, whereas an approx. 16-fold increase was observed for SSO10a-GS4-HWLKV (SEQ ID NO: 46) over DATCS (FIG. 14). Together, this comparison shows that other polypeptides than GCN4, which are capable of forming a dimer, can be used to achieve high affinity toward target.

### Fast Protein Liquid Chromatography—FPLC

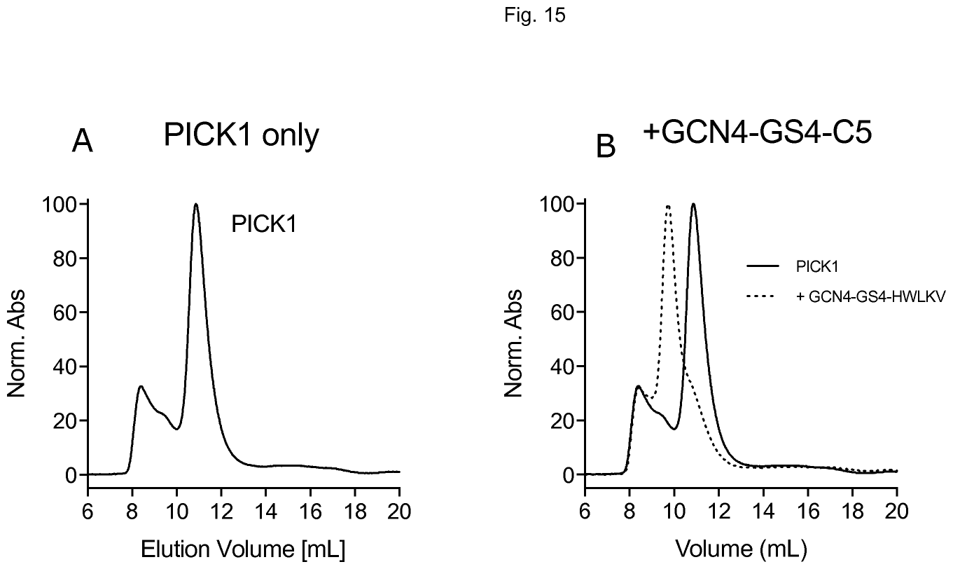

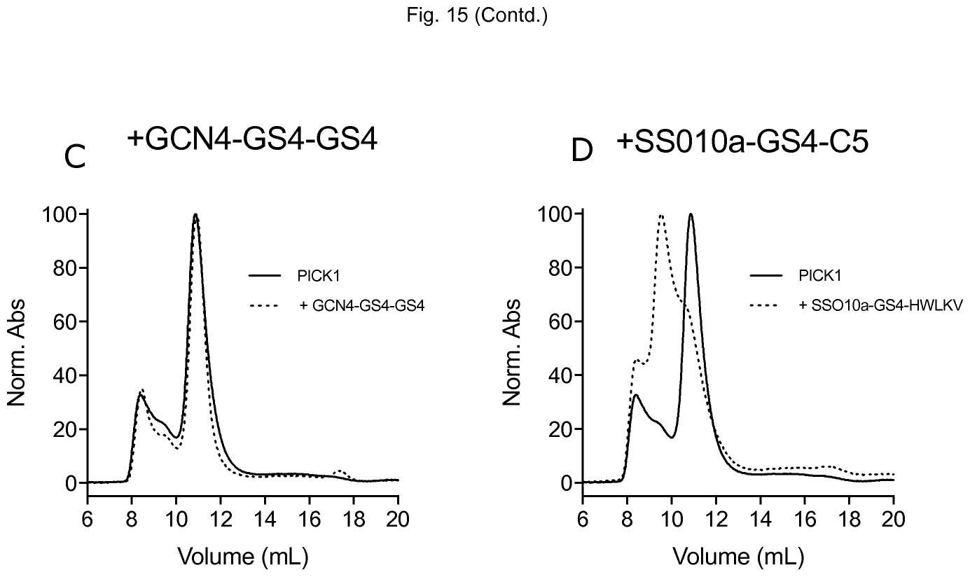

Size exclusion chromatography was done in order to evaluate the in-solution behavior of the SSO10a variant together with PICK1. Elution volumes suggested that SSO10a, like GCN4, induces tetrameric structures of PICK1 (FIGS. 15A,B and D). When incubated with PICK1, the variant GCN4-GS4-GS4 (SEQ ID NO: 38) did not induce a tetrameric state of PICK1 and could be seen at elution volume ˜18 ml (FIG. 15A, C), suggesting that GCN4-GS4-GS4 did not bind to PICK1.

### Example 9

### Oligomerization of PICK1 Upon Binding to GCN4-GS4-C5

As demonstrated in example 2, incubation of PICK1 with GCN4-GS4-C5 in a 4:1 ratio and example 8, incubation of PICK1 with GCN4-GS4-C5 in a 2:1 ratio, resulted in formation of tetrameric configuration of PICK1. A study of the tetramerization was conducted using different PICK1:peptide ratios.

### Materials and Methods

### Size Exclusion Chromatography

PICK1 (40 μM) was incubated in absence or presence of GCN4-GS4-C5, GCN4-GS4-V-to-D or GCN4-GS4-7P14P (20 μM or 400 μM), in 50 mM Tris, 125 mM NaCl, 2 mM DTT, 0.01% TX100, pH 7.4 resulting in a 2:1 and 1:10 PICK1:peptide ratio, respectively. Sample oligomeric composition was validated using SEC (Superdex200 Increase 10/300) measuring the absorbance at 280 nm.

### Results

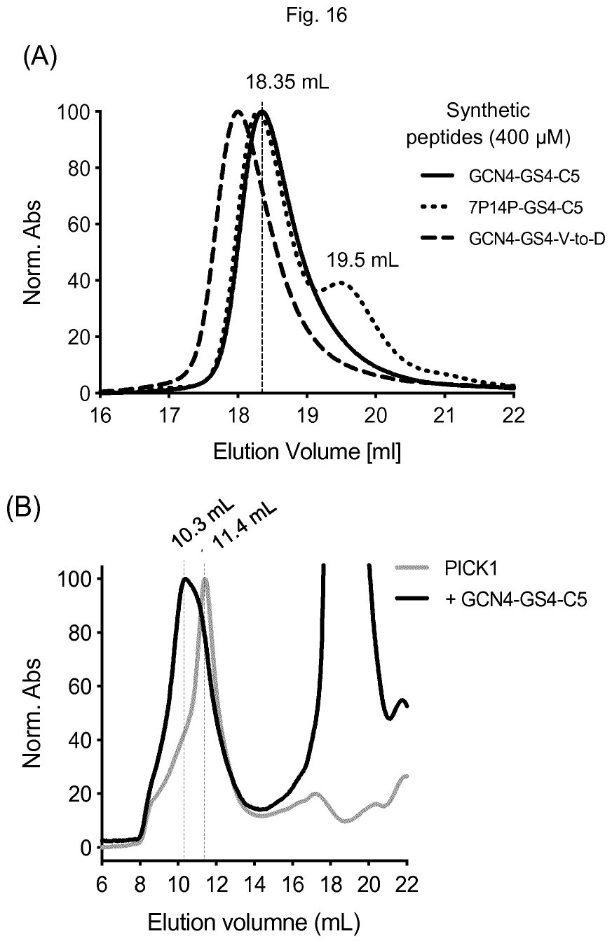

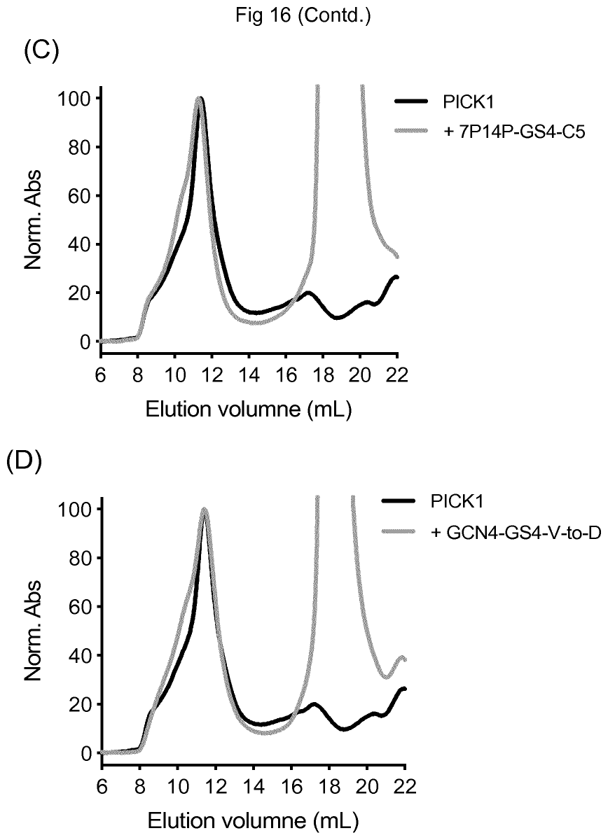

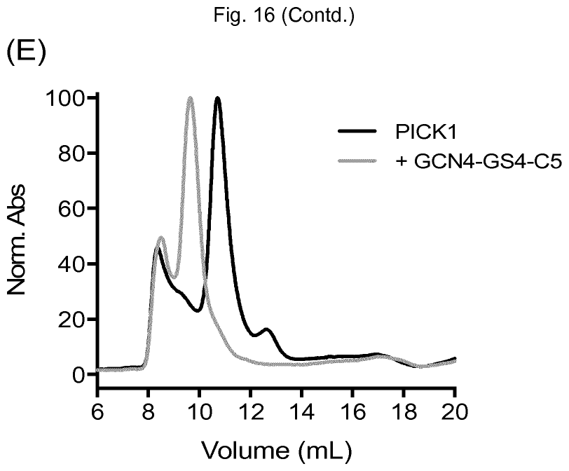

At both ratios, 2:1 and 1:10, GCN4-GS4-C5 effectively shifts PICK1 into a tetrameric configuration, as seen in Table 1 and FIG. 16. Yes' or ‘No’ indicate whether the peptide was able to induce higher oligomerization of PICK1. GCN4-GS4-V-to-D, designed to have the PDZ interacting sequence disrupted, and variant GCN4-GS4-7P14P (i.e. 7P14P-GS4-C5) did not cause PICK1 to oligomerize even at higher concentration.

TABLE 1
Overview of peptides and their ability to induce
higher oligomers of PICK1 assessed through FPLC.

| | Induction of higher oligomers of PICK1 | | | |
|---|---|---|---|---|
| | 1:2 (peptide:PICK1) | | 10:1 (peptide:PICK1) | |
| Synthetic peptide | Yes | No | Yes | No |
| GCN4-GS4-C5 | X | | X | |
| 7P14P-GS4-C5 | | X | | X |
| GCN4-GS4-V-to-D | | X | | X |

Upon incubation of PICK1 (40 μM) with the GCN4-GS4-C5 (20 μM (FIG. 16E) or 400 μM (FIG. 16B)), PICK1 shifted fully from an elution at 11.7 ml (figure xx), which represents the dimeric form of the protein, to an elution at ˜10 ml indication formation of the tetrameric configuration of PICK1 critical for obtaining high affinity binding. GCN4-GS4-V-to-D (400 μM), designed to have the PDZ interacting sequence disrupted, did not cause PICK1 to oligomerize (FIG. 16C). Finally, PICK1 (40 μM) incubated with GCN4-GS4-7P14P (400 μM) also eluted at 11.7 ml (FIG. 16D).

### Example 10

### Efficacy Study of AA V8-GCN4-GS4-C5 and AA V8-GCN4(7P14P)-GS4-C5 in a Model of Inflammatory Pain.

Assessment of the efficacy of a single i.t. administration of AAV8-hSyn-HA-GCN4p1-GS4-C5-WPREpA (AAV8-GCN4-GS4-C5) or AAV8-hSyn-HA-GCN4p1(7P14P)-GS4-C5-WPREpA (AAV8-GCN4(7P14P)-GS4-C5) as compared to AAV8-hSyn-tdTomato-WPREpA to relief inflammatory pain induced by administration of Complete Freund's Adjuvant (CFA).

### Materials and Methods

Animals; 6-9 male C57BL6/N mice (SPF status, Janvier, France) of 8 weeks of age at beginning of experiment were used in each group. Mice were allowed at least 7 days of habituation to our facility before initiation of experiment. Mice were group-housed in IVC-cages in a temperature-controlled room maintained on a 12:12 light:dark cycle (lights on at 6 AM) and allowed access to standard rodent chow and water ad libitum.

Virus; Mice were injected with one of three viruses; AAV8-hSyn-HA-GCN4p1-GS4-C5-WPREpA (n=6 mice), AAV8-hSyn-HA-GCN4p1(7P14P)-GS4-C5-WPREpA (n=6 mice) or the control virus, AAV8-hSyn-tdTomato-WPREpA (n=9 mice). All three viruses were diluted (final titer 2.2E+12 vg/mL, 2.1E+12 vg/mL, and 2.1E+12 vg/mL, respectively) in Dulbecco's Phosphate-Buffered Saline prior to the injection. The virus was delivered by i.t. administration in a volume of 7 μL to mice under isofluorane anesthesia using a 10 pL Hamilton syringe and 30G, 20 mm long, 11 angle tip needle inserted in the intervertebral space between L5/L6 3 weeks prior to the von Frey test. The correct position of the needle was assured by a typical flick of the tail.

Induction of inflammatory pain; On day 19 after virus injection, inflammatory pain was induced by the use of Complete Freund's adjuvant (CFA). Mice were placed under very light isoflurane anesthesia. The right hind paw of the mice was sterilized with ethanol, and 5 μL of CFA was injected intraplantar to the right hind paw with an insulin needle. Mice woke up within seconds of being removed from the isoflurane, and were left for 48 hours while inflammatory pain developed. The development and level of mechanical hyperalgesia/allodynia was determined in the affected hind paws 2, 5, 11 and 12 days after the CFA procedure by using Von Frey filaments ranging from 0.04 to 2 g. The filaments are applied to the underside of the paw after the mouse has settled into a comfortable position within a restricted area that has a perforated floor. The filaments are calibrated to flex when the set force is applied to the paw. Filaments are presented in order of increasing stiffness, until a paw withdrawal is detected. In the current experiments filaments in ascending order were applied to the central part of the hind paws. Each Von Frey hair was applied five times over a total period of 30 seconds and the mouse's reaction was assessed after each application; the threshold for a positive test was set at 3 trials, which evoked responses out of a maximum of 5 trials. A positive pain reaction is defined as sudden paw withdrawal, flinching and/or paw licking induced by the filament.

The non-injected left hind paw was used as an unaffected control (contra).

Measuring the effect of the viral constructs on pain threshold; The pain threshold of the mice was measured before virus injection (no significant difference between groups), before CFA injection (no significant difference between groups), and 2 and 5 days after CFA injection, where hyperalgesia is normally observed in the CFA-injected paw of the animals. This was indeed the case for AAV8-tdTomato injected control mice.

Reinstatement of hyperalgesia; On day 11 after CFA administration, remission phase was confirmed by testing the pain threshold of the animals with von Frey filament, to make sure their apparent pain threshold being back to the pre-injury level. Mice were then injected s.c. with 3 mg/kg naltrexone (NTX), which is a p-opioid receptor (MOR) inverse agonist with a half-life of 2-4 hrs unmasking the latent sensitization of the animals. The pain threshold of the animals was measured after 60 min and 24 hours of the NTX injection.

### Results

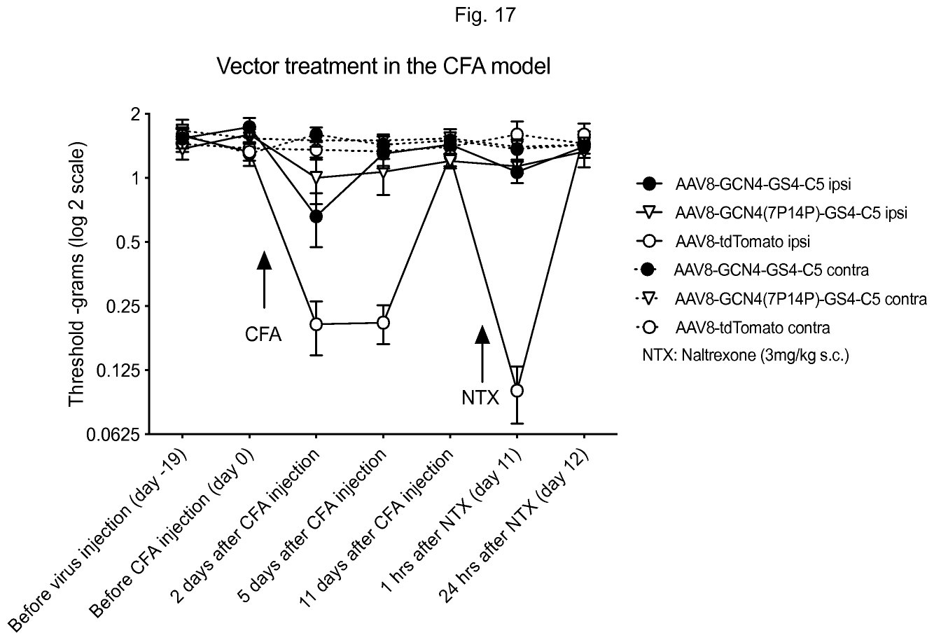

Both AAV8-hSyn-HA-GCN4p1-GS4-C5-WPREpA and AAV8-hSyn-HA-GCN4p1(7P14P)-GS4-C5-WPREpA efficiently relief pain in the CFA model of inflammatory pain. Data was analysed by two-way ANOVA (interaction: F(6,51)=4.203 p=0.0016; Time: F(3,51)=28.40 p<0.0001; Treatment: F(2,17)=11.12 p=0.0008). Post-Bonferroni analysis revealed significant pain relief at both day 2 and 5 post CFA injection (7P14P_{day2} p<0.0001; 7P14P_{day5} p=0.0003; GCN4_{day2} p=0.07; GCN4_{day5} p=0.0008), when comparing pain threshold to the tdTomato control (FIG. 17). Furthermore, both treatments prevented latent sensitization in the CFA model of inflammatory pain. Data was analysed by two-way ANOVA (interaction: F(4,26)=5.237 p=0.0031; Time: F(2,26)=16.08 p<0.0001; Treatment: F(2,13)=5.662 p=0.017). Post-Bonferroni analysis revealed prevention of latent sensitization at 1 hour after naltrexone injection (7P14P_{1hrpostNTX} p<0.0001; GCN4_{1hrpostNTX} p<0.0001), when comparing pain threshold to the tdTomato control (FIG. 17).

### Conclusion

This experiment demonstrates that treatment with a viral construct expressing GCN4-GS4-C5 and 7P14P-GS4-C5 are capable of inducing pain relief in a model of inflammatory pain.

### Example 11

### Treatment of Alzheimer's Disease

Alzheimer's disease (AD) is a devastating neurological disorder with 36 million new cases each year with no cure and no drugs that can delay its progression. An early symptom found in brain tissue is synapse loss, which leads to cognitive impairment, and can be correlated with elevation in soluble amyloid beta (AR) levels. These pathological events occur prior to neuronal death, suggesting that treatment strategies that can prevent synapse loss as an early target may provide a better prognosis for AD therapy than simply altering synaptic transmission. Recent evidences suggest that protein interacting with C kinase 1 (PICK1) is required for AR to weaken and eliminate synapses. In PICK1 deficient mice (KO), AR fails to reduce synapses, and similar results have been obtained with small molecule inhibitors against PICK1 in neuronal cultures. Soluble AR enhances the internalization of AMPA receptors through a GluA2-dependent mechanism. Importantly, the synaptic trafficking of the AMPA GluA2 subunit is controlled by PICK1 that can bind to the C-terminus of the GluA2 subunit via the PICK1-PDZ domain. Collectively, these evidences suggest that if PICK1 can be blocked, then AR will fail to induce synapse loss, and ultimately neuronal cell death. Thus, selective PICK1-GluA2 inhibitors may prove useful for the treatment of AD.

We therefore rationalize that the viral vector treatment of the present disclosure (e.g. AAV8-hSyn-HA-GCN4-GS4-C5-WPREpA) can be relevant for AD, not for prevention, but as an early life-long intervention that can significantly delay the progression of AD e.g. in subjects genetically disposed to develop AD.

### Materials and Methods

Male adult wistar rats (n=20, Charles River) weighing 225-250 g at the beginning of the experiment were used, and housed on a 12 h day/night cycle with ad libitum access to food and water. The viral vectors were produced in-house, and diluted with Dulbecco's Phosphate Buffer prior to injection to reach a final titer of 3 x1012 vg/ml. A total of 2 μl AAV vector was injected into each hippocampus (2 μl in each side; 500 nl per injection site). Amyloid-β 25-35 (3 mg/ml; Bacher AG, Switzerland) was bilateral injected into the lateral ventricles (5 μl each side). Animals were kept on isoflurane anesthesia throughout the surgical procedures.

Coordinates for amyloid-β deposit (in mm): AP −1.0; ML+/−1.5; DV+4.1

Coordinates for vector injection (in mm) for dorsal hippocampus: AP −3.3; ML+/−2.2;

DV −3.3/−2.6; for ventral hippocampus: AP −4.9; ML+/−5.2; DV −6.3/−3.7.

For the Morris Water Maze a circular tank with a diameter of 160 cm and a height of 60 cm was used. It was filled with water up to 20 cm below the top and had a temperature of 21° C. The tank was divided into four quadrants and cues were put on the walls for orientation. A rescue platform (10 cm of diameter) was placed 1.5 cm below the water level in the tank. The rats were placed in different locations of the maze and the time they needed to find the platform was counted. If the rats did not find the platform within 90 seconds, they were guided to the platform. The rats were allowed to sit at the platform for 20 seconds. A break of 20 seconds followed before the trail was repeated with a different starting point for two more times.

### Results

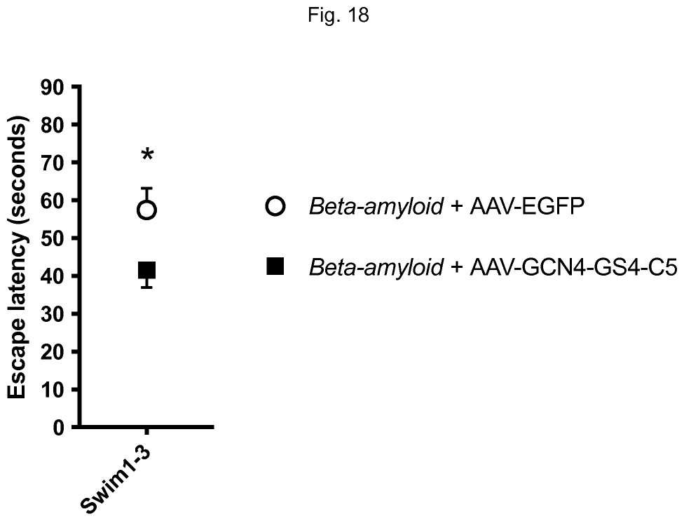

To obtain proof-of-concept for a learning protective effect of our vector treatment, all rats were bilateral injected with Amyloid-β into the lateral ventricles. Such deposit is known to give rise to cognitive deficits. During the same surgical procedure, rats were injected into the hippocampus with AAV8-hSyn-HA-GCN4-GS4-C5-WPREpA (n=10) or a control vector, AAV8-hSyn-EGFP-WPREpA (n=10). Three weeks after, rats were trained in the Morris Water Maze to assess their spatial learning ability (FIG. 18). Rats treated with the recombinant GCN4-GS4-C5 peptide learned significantly faster where to find the hidden platform (P<0.05, t-test).

### Example 12

AAV8-GCN4(7P14P)-GS4-C5 treatment after nerve injury completely reverse mechanical hyperalgesia in the mouse SNI model.

### Materials and Methods

### Mouse Spared Nerve Injury (SNI) Model

The present example was performed as described in example 5, but with a few exceptions, as explained in the following. After dilution, the viral titers of AAV8-hSyn-HA-GCN4p1(7P14P)-GS4-C5-WPREpA and AAV8-hSyn-tdTomato-WPREpA were 2.14E+12 vg/mL and 2.12E+12 vg/mL, respectively. Von Frey was tested on multiple occasions; before SNI, on day −2 and −1; 2 day after SNI (prior to virus administration); and tested 7, 14, 21, 28, 35, and 43 days after SNI. Virus was administered intrathecally 2 days after SNI (and after Von Frey tested the same day).

### Results:

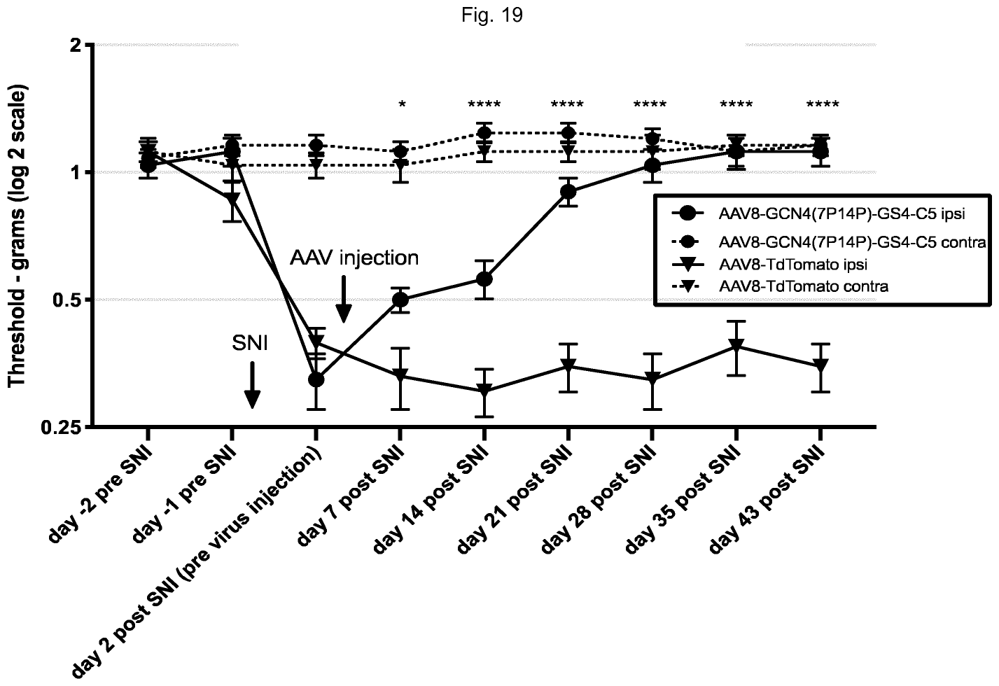

AAV8-hSyn-HA-GCN4(7P14P)-GS4-C5 treatment after injury completely reverse mechanical hyperalgesia in the mouse SNI model (FIG. 19). Mice were injected intrathecally with AAV8-hSyn-tdTomato-WPRE-pA (n=10, serving as a control vector) or AAV8-hSyn-HA-GCN4p1(7P14P)-GS4-C5-WPRE-pA (n=10) 2 days after SNI. Von Frey testing revealed complete pain relief for AAV8-hSyn-HA-GCN4p1(7P14P)-GS4-C5 treated mice on day 7, day 14, day 21, day 28, day 35, day 43 post SNI, as compared to its own intact paw (contra), and as compared to control animals (tdTomato ipsi and tdTomato contra). Data are presented as mean±SEM and were analyzed using a 2-way ANOVA with Tukey's post-hoc test, F (24, 324)=8,644, p<0.0001.

Sequences
DNA sequence
SEQ ID NO: 1 (HA-GCN4p1-gl4-DATC5)
atgtatccgtatgatgtgccggattatgcgggcag
ccgcatgaaacagctggaagataaagtggaagaac
tgctgagcaaaaactatcatctggaaaacgaagtg
gcgcgcctgaaaaaactggtgggcggcggcggcag
ccattggctgaaagtgtaa
This sequence contains a 5′ start
codon (atg) and 3′ stop codon
(taa), but could be any
stop codons (TTA, TGA, TAG).
SEQ ID NO: 2: HA-tag
tatccgtatgatgtgccggattatgcg
SEQ ID NO: 3: WT GCN4 PDB:2ZTA
(GCN4p1 fragment) (including
linker to connect HA to GCN4p1)
ggcagccgcatgaaacagctggaagataaagtgga
agaactgctgagcaaaaactatcatctggaaaacg
aagtggcgcgcctgaaaaaactggtg
SEQ ID NO: 4: gLinker region (gl4 or GS4)
ggcggcggcggcagc
SEQ ID NO: 5 DATC5 (C5)
cattggctgaaagtg.
SEQ ID NO: 13:
(HA-GCN4p1(7P14P)-gl4-DATC5)
Atgtatccgtatgatgtgccggattatgcgggcag
ccgcatgaaacagctggaaccgaaagtggaagaac
tgctgccgaaaaactatcatctggaaaacgaagtggcgcgcct
gaaaaaactggtgggcggcggcggcagccattggc
tgaaagtgtaa
This sequence contains a 5′ start codon
(atg) and 3′ stop codon (taa),
but could be any stop codons (TTA, TGA, TAG).
SEQ ID NO: 14 (HA-GCN4p1-gl4-IETDV)
atgtatccgtatgatgtgccggattatgcgggcag
ccgcatgaaacagctggaagataaagtggaagaac
tgctgagcaaaaactatcatctggaaaacgaagtg
gcgcgcctgaaaaaactggtgggcggcggcggcag
catcgagaccgacgtgtaa
This sequence contains a 5′ start codon
(atg) and 3′ stop codon (taa),
but could be any stop codons (TTA, TGA, TAG).
SEQ ID NO: 15
(HA-GCN4p1(7P14P)-gl4-IETDV)
atgtatccgtatgatgtgccggattatgcgggcag
ccgcatgaaacagctggaaccgaaagtggaagaac
tgctgccgaaaaactatcatctggaaaacgaagtg
gcgcgcctgaaaaaactggtgggcggcggcggcag
catcgagaccgacgtgtaa
This sequence contains a 5′ start codon
(atg) and 3′ stop codon (taa),
but could be any stop codons
(TTA, TGA, TAG).
SEQ ID NO: 16
(HA-GCN4p1-gl4-RRTTPV)
atgtatccgtatgatgtgccggattatgcgggcag
ccgcatgaaacagctggaagataaagtggaagaac
tgctgagcaaaaactatcatctggaaaacgaagtg
gcgcgcctgaaaaaactggtgggcggcggcggcag
cagaagaaccacccctgtgtaa
This sequence contains a 5′ start codon
(atg) and 3′ stop codon (taa), but
could be any stop codons (TTA, TGA, TAG).
SEQ ID NO: 17
(HA-GCN4p 1(7P14P)-gl4-RRTTPV)
atgtatccgtatgatgtgccggattatgcgggcag
ccgcatgaaacagctggaaccgaaagtggaagaac
tgctgccgaaaaactatcatctggaaaacgaagtg
gcgcgcctgaaaaaactggtgggcggcggcggcag
cagaagaaccacccctgtgtaa
This sequence contains a 5′ start codon
(atg) and 3′ stop codon (taa),
but could be any stop codons (TTA, TGA, TAG).
SEQ ID NO: 18 (HA-GCN4p1-gl4-YKQTSV)
atgtatccgtatgatgtgccggattatgcgggcag
ccgcatgaaacagctggaagataaagtggaagaac
tgctgagcaaaaactatcatctggaaaacgaagtg
gcgcgcctgaaaaaactggtgggcggcggcggcag
ctacaagcagaccagcgtgtaa
This sequence contains a 5′ start codon
(atg) and 3′ stop codon (taa),
but could be any stop codons
(TTA, TGA, TAG).
SEQ ID NO: 19
(HA-GCN4p1(7P14P)-gl4-YKQTSV)
atgtatccgtatgatgtgccggattatgcgggcag
ccgcatgaaacagctggaaccgaaagtggaagaac
tgctgccgaaaaactatcatctggaaaacgaagtg
gcgcgcctgaaaaaactggtgggcggcggcggcag
ctacaagcagaccagcgtgtaa
This sequence contains a 5′ start codon
(atg) and 3′ stop codon (taa),
but could be any stop codons
(TTA, TGA, TAG).
SEQ ID NO: 20: (SSO10A-gl4-HWLKV)
atgtatccgtatgatgtgccggattatgcgggcag
cggcgaggagctgctggaggacatcaggaagttca
acgagatgaggaagaacatggaccagctgaaggag
aagatcaacagcgtgctgagcatcaggcagggcgg
cggcggcagccattggctgaaagtgtaa
This sequence contains a 5′ start codon
(atg) and 3′ stop codon (taa),
but could be any stop codons
(TTA, TGA, TAG).
SEQ ID NO: 21: (tdTomato)
atggtgagcaagggcgaggaggtcatcaaagagtt
catgcgcttcaaggtgcgcatggagggctccatga
acggccacgagttcgagatcgagggcgagggcgag
ggccgcccctacgagggcacccagaccgccaagct
gaaggtgaccaagggcggccccctgcccttcgcct
gggacatcctgtccccccagttcatgtacggctcc
aaggcgtacgtgaagcaccccgccgacatccccga
ttacaagaagctgtccttccccgagggcttcaagt
gggagcgcgtgatgaacttcgaggacggcggtctg
gtgaccgtgacccaggactcctccctgcaggacgg
cacgctgatctacaaggtgaagatgcgcggcacca
acttcccccccgacggccccgtaatgcagaagaag
accatgggctgggaggcctccaccgagcgcctgta
cccccgcgacggcgtgctgaagggcgagatccacc
aggccctgaagctgaaggacggcggccactacctg
gtggagttcaagaccatctacatggccaagaagcc
cgtgcaactgcccggctactactacgtggacacca
agctggacatcacctcccacaacgaggactacacc
atcgtggaacagtacgagcgctccgagggccgcca
ccacctgttcctggggcatggcaccggcagcaccg
gcagcggcagctccggcaccgcctcctccgaggac
aacaacatggccgtcatcaaagagttcatgcgctt
caaggtgcgcatggagggctccatgaacggccacg
agttcgagatcgagggcgagggcgagggccgcccc
tacgagggcacccagaccgccaagctgaaggtgac
caagggcggccccctgcccttcgcctgggacatcc
tgtccccccagttcatgtacggctccaaggcgtac
gtgaagcaccccgccgacatccccgattacaagaa
gctgtccttccccgagggcttcaagtgggagcgcg
tgatgaacttcgaggacggcggtctggtgaccgtg
acccaggactcctccctgcaggacggcacgctgat
ctacaaggtgaagatgcgcggcaccaacttccccc
ccgacggccccgtaatgcagaagaagaccatgggc
tgggaggcctccaccgagcgcctgtacccccgcga
cggcgtgctgaagggcgagatccaccaggccctga
agctgaaggacggcggccactacctggtggagttc
aagaccatctacatggccaagaagcccgtgcaact
gcccggctactactacgtggacaccaagctggaca
tcacctcccacaacgaggactacaccatcgtggaa
cagtacgagcgctccgagggccgccaccacctgtt
cctgtacggcatggacgagctgtacaagtaa
This sequence contains a 5′ start codon
(atg) and 3′ stop codon (taa),
but could be any other stop codons
Amino acid sequence
SEQ ID NO: 6
(GCN4p1-GS4-DATC5)
RMKQLEDKVEELLSKNYHLENEVARLKKLV-GGGGS-HWLKV
SEQ ID NO: 7: HA-tag
YPYDVPDYA
SEQ ID NO: 8: WT GCN4 PDB:2ZTA
(GCN4p1 fragment)
RMKQLEDKVEELLSKNYHLENEVARLKKLV
SEQ ID NO: 9:
gLinker region (GS4 or gl4)
GGGGS
SEQ ID NO: 10 (DATC5 or C5)
HWLKV.
SEQ ID NO: 11
Z₁Z₂Z₃Z₄Z₅.
wherein:
Z₁ is a proteogenic or non-proteogenic
amino acid, preferably H, L, V, 1,
A; or is absent;
Z₂ is a proteogenic or non-proteogenic
amino acid, preferably W, F, S, T;
or is absent;
Z₃ is a proteogenic or non-proteogenic
amino acid, preferably L, V, 1,
F, A, Y;
Z₄ is a proteogenic or non-proteogenic
amino acid, preferably K, R, S, T;
and
Z₅ is V, I, L or C.
SEQ ID NO: 12
L-[X]6- L-[X]6-L-[X]6-L.
wherein X is individually selected from
any proteinogenic or non-proteinogenic
amino acid residue.
SEQ ID NO: 22
Z₁Z₂Z₃Z₄Z₅.
wherein:
Z₁ is H, L, V, I, A; or is absent;
Z₂ is W, F, S, T; or is absent;
Z₃ is L, V, I, F, A or Y;
Z₄ is K, R, S or T; and
Z₅ iS V, I, L or C.
SEQ ID NO: 23
Z₁Z₂Z₃Z₄Z₅.
wherein:
Z₁ is H, L, V, I or A;
Z₂ iS W, F, S or T;
Z₃ is L, V, I, F, A or Y;
Z₄ is K, R, S or T; and
Z₅ iS V, I, L or C.
SEQ ID NO: 24
Z₁Z₂Z₃Z₄Z₅.
wherein:
Z₁ is H, V, I or A;
Z₂ iS W, F or S;
Z₃ is L, V or I;
Z₄ is K, R, S; and
Z₅ is V or C.
SEQ ID NO: 25
Z₁Z₂Z₃Z₄Z₅.
wherein:
Z₁ is H, V or A;
Z₂ is W or S;
Z₃ is L, V or I;
Z₄ is K or R; and
Z₅ is V.
SEQ ID NO: 26
(HA-GCN4p1-GS4-DATC5)
YPYDVPDYAGSRMKQLEDKVEELLSKNYH
LENEVARLKKLV-GGGGS-HWLKV
SEQ ID NO: 27
(GCN4p1(7P14P) or 7P14P)
RMKQLEPKVEELLPKNYHLENEVARLKKLV
SEQ ID NO: 28
(GCN4p1(7P14P)-GS4-DATC5 or
7P14P-GS4-05 or GCN4-GS4-7P14P)
RMKQLEPKVEELLPKNYHLENEVARLKKLV-GGGGS-
HWLKV
SEQ ID NO: 29
IETDV
SEQ ID NO: 30
(GCN4-GS4-IETDV)
RMKQLEDKVEELLSKNYHLENEVARLKKLV-GGGGS-IETDV
SEQ ID NO: 31
(GCN41p(7P14P)-GS4-IETDV)
RMKQLEPKVEELLPKNYHLENEVARLKKLV-GGGGS-IETDV
SEQ ID NO: 32
RRTTPV
SEQ ID NO: 33
(GCN4p1-GS4-RRTTPV)
RMKQLEDKVEELLSKNYHLENEVARLKKLV-GGGGS-RRTTPV
SEQ ID NO: 34
(7P14P-GS4-RRTTPV)
RMKQLEPKVEELLPKNYHLENEVARLKKLV-GGGGS-RRTTPV
SEQ ID NO: 35
YKQTSV
SEQ ID NO: 36 (GCN4p1-GS4-YKQTSV)
RMKQLEDKVEELLSKNYHLENEVARLKKLV-GGGGS-YKQTSV
SEQ ID NO: 37 (7P14P-GS4-YKQTSV)
RMKQLEPKVEELLPKNYHLENEVARLKKLV-GGGGS-YKQTSV
SEQ ID NO: 38 (GCN4-GS4-GS4)
RMKQLEDKVEELLSKNYHLENEVARLKKLV-GGGGS-GGGGS
SEQ ID NO: 39 (GCN4-GS4-V-to-D)
RMKQLEDKVEELLSKNYHLENEVARLKKLV-GGGGS-HWLKD
SEQ ID NO: 40
KVDSV
SEQ ID NO: 41
GKDYV
SEQ ID NO: 42
RKDYV
SEQ ID NO: 43
TAEMF
SEQ ID NO: 44
QEDGA
SEQ ID NO: 45
(α4 helix of the SSO10a protein)
GEELLEDIRKFNEMRKNMDQLKEKINSVLSIRQ
SEQ ID NO: 46:
(SS010a-GS4-HWLKV)
GEELLEDIRKFNEMRKNMDQLKEKINSVLSIR
Q-GGGGS-HWLKV
SEQ ID NO: 47
L-VKAEHG-L-DKVEEQ-L-EVARAK-L
SEQ ID NO: 48 (gLinker3)
GGGS
SEQ ID NO: 49 (gLinker5)
GGGGSG
SEQ ID NO: 50 (gLinker6)
GGGGSGG

### Synonyms

The terms HWLKV, DATC5, and C5 refer to the same peptide having the amino acid sequence of SEQ ID NO: 10.

The terms GCN4, p1GCN4, and GCN4p1 refer to the same peptide having the amino acid sequence of SEQ ID NO: 8.

The terms glinker4, g14, and GS4 refer to the same peptide having the amino acid sequence of SEQ ID NO: 9.

The terms containing 7P14P refers to a peptide comprising the amino acid sequence of SEQ ID: 27

The terms GCN4-GS4-DATC5, GCN4-GS4-C5, GCN4-GS4-HWLKV, GCN4p1-GS4-DATC5, GCN4p1-GS4-C5, and GCN4-GS4-HWLKV, p1GCN4-GS4-05 or GCN4-g14-HWLKV, refer to the same peptide having the sequence of SEQ ID NO: 6

GCN4p1(7P14P)-GS4-DATC5, 7P14P-GS4-C5 and GCN4-GS4-7P14P refer to the same peptide having the sequence of SEQ ID NO: 28.

### REFERENCES

ALFONSO, S., et al., Synapto-depressive effects of amyloid beta require PICK1. Eur J Neurosci, 2014. 39(7): p. 1225-33.
ATIANJOH, F. E., et al., Spinal cord protein interacting with C kinase 1 is required for the maintenance of complete Freund's adjuvant-induced inflammatory pain but not for incision-induced post-operative pain. Pain, 2010. 151(1): p. 226-34.
BACH et al., PNAS, 109(9):3317-22, 2012
CHEN et al., The Hyperthermophile Protein SSO10a is a Dimer of Winged Helix DNA-binding Domains Linked by an Antiparallel Coiled Coil Rod, J. Mol. Biol., 2004 341, 73-91
CHEN, S. R., et al., Nerve injury increases GluA2-lacking AMPA receptor prevalence in spinal cords: functional significance and signaling mechanisms. J Pharmacol Exp Ther, 2013. 347(3): p. 765-72.
CLEM, R. L., V. Anggono, and R. L. Huganir, PICK1 regulates incorporation of calcium-permeable AMPA receptors during cortical synaptic strengthening. J Neurosci, 2010. 30(18): p. 6360-6.
CONRAD, K. L., et al., Formation of accumbens GluR2-lacking AMPA receptors mediates incubation of cocaine craving. Nature, 2008. 454(7200): p. 118-21.
DIXON, R. M., J. R. Mellor, and J. G. Hanley, Pick1-mediated glutamate receptor subunit 2 (GLUR2) trafficking contributes to cell death in oxygen/glucose deprived hippocampal neurons. Journal of Biological Chemistry, 2009.
GANGADHARAN, V., et al., Peripheral calcium-permeable AMPA receptors regulate chronic inflammatory pain in mice. J Clin Invest, 2011. 121(4): p. 1608-23.
GARRY, E. M., et al., Specific involvement in neuropathic pain of AMPA receptors and adapter proteins for the GluR2 subunit. Mol.Cell Neurosci., 2003. 24(1): p. 10-22.
GONZARLEZ et al., 1996. Buried polar residues and structural specificity in the GCN4 leucine zipper. Nat. Struct Biol (12):1011-8.
HE, J., et al., PICK1 inhibits the E3 ubiquitin ligase activity of Parkin and reduces its neuronal protective effect. Proc Natl Acad Sci USA, 2018. 115(30): p. E7193-E7201.
JI, R. R., et al., Central sensitization and LTP: do pain and memory share similar mechanisms? Trends Neurosci, 2003. 26(12): p. 696-705.
KANDASAMY, R. and T. J. Price, The pharmacology of nociceptor priming. Handb Exp Pharmacol, 2015. 227: p. 15-37.
LEDER L. et al., 1995. Spectroscopic, calorimetric, and Kinetic Demonstration of Conformational Adaptation in Peptide-Antibody Recognition. Biochemistry 34, 16509-16518.
LEE J. H. et al., 2014. Protein grafting of p53TAD onto a leucine zipper scaffold generates a potent HDM dual inhibitor. Nat Commun. 5:3814
LORGEN, JØ., PICK1 facilitates lasting reduction of GluA2 concentration in the hippocampus during chronic epilepsy. Epilepsy Res, 2017. 137: 25-32
LUMB, K. J. 1994, Subdomain folding of the coiled coil leucine zipper from the bZIP transcriptional activator GCN4. Biochemistry 33, 7361-7367.
LUSCHER, C. and R. C. Malenka, Drug-evoked synaptic plasticity in addiction: from molecular changes to circuit remodeling. Neuron, 2011. 69(4): p. 650-63.
MADSEN, K. L. et al., 2005. Molecular determinants for the complex binding specificity of the PDZ domain in PICK1. Journal of Biological Chemistry 280, 20539-20548.
MARCOTTE, D. J., et al., Lock and chop: A novel method for the generation of a PICK1 PDZ domain and piperidine-based inhibitor co-crystal structure. Protein Sci, 2018. 27(3): p. 672-680.
MOLLER, A. R., Tinnitus and pain. Prog Brain Res, 2007. 166: p. 47-53.
OSHABEN K. M. et al., 2012. The native GCN4 leucine-zipper domain does not uniquely specify a dimeric oligomerization state. Biochemistry 51(47):9581-91
O'SHEA E K, et al., 1991, X-ray structure of the GCN4 leucine zipper, a two-stranded, parallel coiled coil. Science 25 Oct. 1991: Vol. 254, Issue 5031, pp. 539-544, DOI: 10.1126/science.1948029
PEKER, S. and A. Sirin, Parallels between phantom pain and tinnitus. Med Hypotheses, 2016. 91: p. 95-97.
RICHNER et al., 2011. The spared nerve injury (SNI) model of induced mechanical allodynia in mice. J Vis Exp. (54). pii: 3092.
TOTO, A. et al Protein Engineering, Design and Selection, Volume 29, Issue 5, 2016, P. 169-175
SAINLOS et al. Nat. Chem. Biol. 7, 81-91, 2011
SØRENSEN et al., 2016, A robust activity marking system for exploring active neuronal ensembles. eLife 2016; 5:e13918
VANNESTE, S., W. T. To, and D. De Ridder, Tinnitus and neuropathic pain share a common neural substrate in the form of specific brain connectivity and microstate profiles. Prog Neuropsychopharmacol Biol Psychiatry, 2019. 88: p. 388-400.
VIKMAN, K. S., B. K. Rycroft, and M. J. Christie, Switch to Ca2+-permeable AMPA and reduced NR2B NMDA receptor-mediated neurotransmission at dorsal horn nociceptive synapses during inflammatory pain in the rat. J Physiol, 2008. 586(2): p. 515-27.
WANG, W., et al., Preserved acute pain and impaired neuropathic pain in mice lacking protein interacting with C Kinase 1. Mol Pain, 2011. 7: p. 11.
WOLF, M. E. and C. R. Ferrario, AMPA receptor plasticity in the nucleus accumbens after repeated exposure to cocaine. Neurosci Biobehav Rev, 2010. 35(2): p. 185-211.
WOOLF, C. J. and M. W. Salter, Neuronal plasticity: increasing the gain in pain. Science, 2000. 288(5472): p. 1765-9.
ZENG et al. Cell. 2016 Aug. 25; 166(5):1163-1175.
ZHANG et al. Protein interacting with C alpha kinase 1 (PICK1) is involved in promoting tumor growth and correlates with poor prognosis of human breast cancer. Cancer Sci. 2010 June; 101(6):1536-42.

1.-32. (canceled)

33. A polynucleotide comprising a sequence encoding upon expression a polypeptide comprising:
a) a first polypeptide part comprising or consisting of an amino acid sequence capable of forming a homodimer with an identical first polypeptide part; and
b) a second polypeptide part comprising or consisting of an amino acid sequence selected from the group consisting of Class I PDZ domains binding motifs (PBM), Class II PBM and Class III PBM,
wherein the first and the second polypeptide parts are optionally operably linked via a linker.

34. The polynucleotide according claim 33, wherein said polynucleotide comprises a sequence encoding upon expression:
a) a first polypeptide part comprising or consisting of an amino acid sequence of the general formula (I): L-[X]6-L-[X]6-L-[X]6-L (SEQ ID NO:12), wherein X is individually selected from any proteinogenic or non-proteinogenic amino acid residue; and
b) a second polypeptide part comprising or consisting of an amino acid sequence of the general formula (II):
(SEQ ID NO: 11)
Z₁Z₂Z₃Z₄Z₅;
wherein:
Z₁ is a proteogenic or non-proteogenic amino acid, preferably H, L, V, I, A; or is absent;
Z₂ is a proteogenic or non-proteogenic amino acid, preferably W, F, T, S; or is absent;
Z₃ is a proteogenic or non-proteogenic amino acid, preferably L, V, I, F; A, Y;
Z₄ is a proteogenic or non-proteogenic amino acid, preferably K, R, T, S; and
Z₅ is V, I, L or C.

35. The polynucleotide according to claim 33, wherein the first polypeptide part is selected from the group consisting of GCN4p1, GCN4p1(7P14P), and SSO10a.

36. The polynucleotide according to claim 33, wherein the first polypeptide part comprises an alpha helix.

37. The polynucleotide according to claim 33, wherein the homodimer of the first polypeptide part is formed as a coiled coil.

38. The polynucleotide according claim 33, wherein the second polypeptide is consisting of or comprising a sequence selected from the group consisting of Σ-¥-Ψ, Ψ-¥-Ψ, and Φ-¥-Ψ, wherein
Σ is Thr or Ser;
¥ is any proteinogenic amino acid;
Ψ is any hydrophobic amino acid; and
Φ is Asp or Glu.

39. The polynucleotide according to claim 33, wherein the second polypeptide part consists of or comprises IETDV, RRTTPV, or YKQTSV.

40. The polynucleotide according to claim 33, wherein the second polypeptide part consists of or comprises the sequence HWLKV.

41. The polynucleotide according to claim 33, wherein the second polypeptide part consists of or comprises KVDSV, GKDYV, RKDYV, TAEMF or QEDGA.

42. The polynucleotide according to claim 33, wherein the second polypeptide part is consisting of or comprising an amino acid sequence of the general formula (II):
(SEQ ID NO: 23)
Z₁Z₂Z₃Z₄Z₅;
wherein:
Z₁ is H, L, V, I, A;
Z₂ is W, F, T, S;
Z₃ is L, V, I, F, A, Y;
Z₄ K, R, T, S; and
Z₅ is V, I, L or C.

43. The polynucleotide according to claim 33, wherein the second polypeptide part is consisting of or comprising an amino acid sequence of the general formula (II):
(SEQ ID NO: 25)
Z₁Z₂Z₃Z₄Z₅;
wherein:
Z₁ is H, V, A;
Z₂ is W or S;
Z₃ is L, V, I;
Z₄ K or R; and
Z₅ is V.

44. The polynucleotide according to claim 33, further comprising the linker and wherein the linker is a glycine serine linker selected from the group consisting of GGGGS (glinker4), GGS (gLinker2), GGGS (gLinker3), GGGGSG (gLinker5), GGGGSGG (gLinker6).

45. The polynucleotide according claim 33, wherein the second polypeptide binds to a PDZ domain.

46. The polynucleotide according claim 33, wherein the second polypeptide is capable of inhibiting the interaction between AMPAR and PICK1, the interaction between cytosolic kinases and PICK1, the interaction between synaptic scaffold proteins and PICK1, the interaction between membrane embedded proteins and PICK1, the interaction between NMDAR and PSD-95, the interaction between membrane embedded proteins and PSD-95, or the interaction between synaptic scaffold proteins and PSD-95.

47. The polynucleotide according to claim 33, wherein polypeptide has a Ki for the PDZ domain below 25 μM.

48. The polynucleotide according to claim 33 further comprising a promoter sequence.

49. An expression vector comprising a polynucleotide sequence encoding upon expression a polypeptide comprising:
a) a first polypeptide part comprising or consisting of an amino acid sequence capable of forming a homodimer with an identical first polypeptide part; and
b) a second polypeptide part comprising or consisting of an amino acid sequence selected from the group consisting of Class I PDZ domains binding motifs (PBM), Class II PBM and Class III PBM,
wherein the first and the second polypeptide parts are operably linked via a linker.

50. The expression vector according to claim 49, wherein the expression vector is an adeno associated vector (AAV).

51. The expression vector according to claim 49, wherein said vector is comprising a sequence encoding upon expression a polypeptide, wherein said polypeptide comprises or consists of the amino acid sequence of GCN4-GS4-C5 (SEQ ID NO: 6), GCN4(7P14P)-GS4-C5 (SEQ ID NO: 28), GCN4-GS4-IETDV (SEQ ID NO: 30), GCN4(7P14P)-GS4-IETDV (SEQ ID NO: 31), GCN4-GS4-RRTTPV (SEQ ID NO: 33), GCN4(7P14P)-GS4-RRTTPV (SEQ ID NO: 34), GCN4-GS4-YKQTSV (SEQ ID NO: 36), GCN4(7P14P)-GS4-YKQTSV (SEQ ID NO: 37), and SSO10A-GS4-C5 (SEQ ID NO: 46).

52. A polypeptide comprising:
a) a first polypeptide part comprising or consisting of an amino acid sequence capable of forming a homodimer with an identical first polypeptide part; and
b) a second polypeptide part comprising or consisting of an amino acid sequence selected from the group consisting of Class I PDZ domains binding motifs (PBM), Class II PBM and Class III PBM,
wherein the first and the second polypeptides are optionally operably linked via a linker.

53. A method for treatment or prevention of a disease and/or disorder associated with maladaptive plasticity and/or transmission comprising administration of a polynucleotide encoding a polypeptide comprising:
a) a first polypeptide part comprising or consisting of an amino acid sequence capable of forming a homodimer with an identical first polypeptide part; and
b) a second polypeptide part comprising or consisting of an amino acid sequence selected from the group consisting of Class I PDZ domains binding motifs (PBM), Class II PBM and Class III PBM,
wherein the first and the second polypeptides are optionally operably linked via a linker, to an individual in need thereof.

54. The method according to claim 53, wherein the disease and/or disorder is selected from the group consisting of pain, drug addiction, ischemia, Alzheimer's disorder, Parkinson's disease, amyotrophic lateral sclerosis, hearing disorders (e.g. tinnitus), migraine, epilepsy, Alzheimer's disease and Parkinson's disease.


SEQUENCE LISTING (50 sequences)

SEQ ID NO: 1 - DNA, 159 bp, artificial sequence
  HA-GCN4p1-gl4-DATC5
atgtatccgtatgatgtgccggattatgcgggcagccgcatgaaacagctggaagataaagtggaagaactgctgagcaaaaactatcatctggaaaacgaagtggcgcgcctgaaaaaactggtgggcggcggcggcagccattggctgaaagtgtaa

SEQ ID NO: 2 - DNA, 27 bp, artificial sequence
  HA-tag
tatccgtatgatgtgccggattatgcg

SEQ ID NO: 3 - DNA, 96 bp, artificial sequence
  GCN4p1 fragment
ggcagccgcatgaaacagctggaagataaagtggaagaactgctgagcaaaaactatcatctggaaaacgaagtggcgcgcctgaaaaaactggtg

SEQ ID NO: 4 - DNA, 15 bp, artificial sequence
  gLinker region (gl4 or GS4)
ggcggcggcggcagc

SEQ ID NO: 5 - DNA, 15 bp, artificial sequence
  DATC5
cattggctgaaagtg

SEQ ID NO: 6 - protein, 40 aa, artificial sequence
  GCN4p1-GS4-DATC5
RMKQLEDKVEELLSKNYHLENEVARLKKLVGGGGSHWLKV

SEQ ID NO: 7 - protein, 9 aa, artificial sequence
  HA-tag
YPYDVPDYA

SEQ ID NO: 8 - protein, 30 aa, artificial sequence
  GCN4p1 fragment
RMKQLEDKVEELLSKNYHLENEVARLKKLV

SEQ ID NO: 9 - protein, 5 aa, artificial sequence
  gLinker region (GS4)
GGGGS

SEQ ID NO: 10 - protein, 5 aa, artificial sequence
  DATC5
HWLKV

SEQ ID NO: 11 - protein, 5 aa, artificial sequence
  Variants of DATC5
  MISC_FEATURE (1)..(1): is a proteogenic or non-proteogenic amino acid, preferably H, L, V, I, A; or is absent
  MISC_FEATURE (2)..(2): is a proteogenic or non-proteogenic amino acid, preferably W, F, S, T; or is absent;
  MISC_FEATURE (3)..(3): is a proteogenic or non-proteogenic amino acid, preferably L, V, I, F, A, Y;
  MISC_FEATURE (4)..(4): is a proteogenic or non-proteogenic amino acid, preferably K, R, S, T
  MISC_FEATURE (5)..(5): is V, I, L or C
XXXXX

SEQ ID NO: 12 - protein, 22 aa, artificial sequence
  general sequence of leucine zipper
  MISC_FEATURE (2)..(7): is individually selected from any proteinogenic or non-proteinogenic amino acid residue
  MISC_FEATURE (9)..(14): is individually selected from any proteinogenic or non-proteinogenic amino acid residue
  MISC_FEATURE (16)..(21): is individually selected from any proteinogenic or non-proteinogenic amino acid residue
LXXXXXXLXXXXXXLXXXXXXL

SEQ ID NO: 13 - DNA, 159 bp, artificial sequence
  HA-GCN4p1(7P14P)-gl4-DATC5
atgtatccgtatgatgtgccggattatgcgggcagccgcatgaaacagctggaaccgaaagtggaagaactgctgccgaaaaactatcatctggaaaacgaagtggcgcgcctgaaaaaactggtgggcggcggcggcagccattggctgaaagtgtaa

SEQ ID NO: 14 - DNA, 159 bp, artificial sequence
  HA-GCN4p1-gl4-IETDV
atgtatccgtatgatgtgccggattatgcgggcagccgcatgaaacagctggaagataaagtggaagaactgctgagcaaaaactatcatctggaaaacgaagtggcgcgcctgaaaaaactggtgggcggcggcggcagcatcgagaccgacgtgtaa

SEQ ID NO: 15 - DNA, 159 bp, artificial sequence
  HA-GCN4p1(7P14P)-gl4-IETDV
atgtatccgtatgatgtgccggattatgcgggcagccgcatgaaacagctggaaccgaaagtggaagaactgctgccgaaaaactatcatctggaaaacgaagtggcgcgcctgaaaaaactggtgggcggcggcggcagcatcgagaccgacgtgtaa

SEQ ID NO: 16 - DNA, 162 bp, artificial sequence
  HA-GCN4p1-gl4-RRTTPV
atgtatccgtatgatgtgccggattatgcgggcagccgcatgaaacagctggaagataaagtggaagaactgctgagcaaaaactatcatctggaaaacgaagtggcgcgcctgaaaaaactggtgggcggcggcggcagcagaagaaccacccctgtgtaa

SEQ ID NO: 17 - DNA, 162 bp, artificial sequence
  HA-GCN4p1(7P14P)-gl4-RRTTPV
atgtatccgtatgatgtgccggattatgcgggcagccgcatgaaacagctggaaccgaaagtggaagaactgctgccgaaaaactatcatctggaaaacgaagtggcgcgcctgaaaaaactggtgggcggcggcggcagcagaagaaccacccctgtgtaa

SEQ ID NO: 18 - DNA, 162 bp, artificial sequence
  HA-GCN4p1-gl4-YKQTSV
atgtatccgtatgatgtgccggattatgcgggcagccgcatgaaacagctggaagataaagtggaagaactgctgagcaaaaactatcatctggaaaacgaagtggcgcgcctgaaaaaactggtgggcggcggcggcagctacaagcagaccagcgtgtaa

SEQ ID NO: 19 - DNA, 162 bp, artificial sequence
  HA-GCN4p1(7P14P)-gl4-YKQTSV
atgtatccgtatgatgtgccggattatgcgggcagccgcatgaaacagctggaaccgaaagtggaagaactgctgccgaaaaactatcatctggaaaacgaagtggcgcgcctgaaaaaactggtgggcggcggcggcagctacaagcagaccagcgtgtaa

SEQ ID NO: 20 - DNA, 168 bp, artificial sequence
  SSO10A- gl4-HWLKV
atgtatccgtatgatgtgccggattatgcgggcagcggcgaggagctgctggaggacatcaggaagttcaacgagatgaggaagaacatggaccagctgaaggagaagatcaacagcgtgctgagcatcaggcagggcggcggcggcagccattggctgaaagtgtaa

SEQ ID NO: 21 - DNA, 1431 bp, artificial sequence
  tdTomato
atggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtaa

SEQ ID NO: 22 - protein, 5 aa, artificial sequence
  Variants of DATC5
  MISC_FEATURE (1)..(1): H, L, V, I, A; or is absent
  MISC_FEATURE (2)..(2): is W, F, S, T; or is absent
  MISC_FEATURE (3)..(3): is L, V, I, F, A, Y
  MISC_FEATURE (4)..(4): is K, R, S, T
  MISC_FEATURE (5)..(5): is V, I, L or C
XXXXX

SEQ ID NO: 23 - protein, 5 aa, artificial sequence
  Variants of DATC5
  MISC_FEATURE (1)..(1): is H, L, V, I, A
  MISC_FEATURE (2)..(2): is W, F, S, T
  MISC_FEATURE (3)..(3): is L, V, I, F, A, Y
  MISC_FEATURE (4)..(4): is K, R, S, T
  MISC_FEATURE (5)..(5): is V, I, L or C
XXXXX

SEQ ID NO: 24 - protein, 5 aa, artificial sequence
  Variants of DATC5
  MISC_FEATURE (1)..(1): is H, V, I, A
  MISC_FEATURE (2)..(2): is W, F, S
  MISC_FEATURE (3)..(3): is L, V, I
  MISC_FEATURE (4)..(4): is K, R, S
  MISC_FEATURE (5)..(5): is V or C
XXXXX

SEQ ID NO: 25 - protein, 5 aa, artificial sequence
  Variants of DATC5
  MISC_FEATURE (1)..(1): is H, V, A
  MISC_FEATURE (2)..(2): is W or S
  MISC_FEATURE (3)..(3): is L, V, I
  MISC_FEATURE (4)..(4): is K or R
  MISC_FEATURE (5)..(5): is V
XXXXX

SEQ ID NO: 26 - protein, 51 aa, artificial sequence
  HA-GCN4p1-GS4-DATC5
YPYDVPDYAGSRMKQLEDKVEELLSKNYHLENEVARLKKLVGGGGSHWLKV

SEQ ID NO: 27 - protein, 30 aa, artificial sequence
  GCN4p1(7P14P)
RMKQLEPKVEELLPKNYHLENEVARLKKLV

SEQ ID NO: 28 - protein, 40 aa, artificial sequence
  GCN4p1(7P14P)-GS4-DATC5
RMKQLEPKVEELLPKNYHLENEVARLKKLVGGGGSHWLKV

SEQ ID NO: 29 - protein, 5 aa, artificial sequence
  Class I PBM
IETDV

SEQ ID NO: 30 - protein, 40 aa, artificial sequence
  GCN4-GS4-IETDV
RMKQLEDKVEELLSKNYHLENEVARLKKLVGGGGSIETDV

SEQ ID NO: 31 - protein, 40 aa, artificial sequence
  GCN4p1(7P14P)-GS4-IETDV
RMKQLEPKVEELLPKNYHLENEVARLKKLVGGGGSIETDV

SEQ ID NO: 32 - protein, 6 aa, artificial sequence
  Class I PBM
RRTTPV

SEQ ID NO: 33 - protein, 41 aa, artificial sequence
  GCN4p1-GS4-RRTTPV
RMKQLEDKVEELLSKNYHLENEVARLKKLVGGGGSRRTTPV

SEQ ID NO: 34 - protein, 41 aa, artificial sequence
  GCN4p1(7P14P)-GS4-RRTTPV
RMKQLEPKVEELLPKNYHLENEVARLKKLVGGGGSRRTTPV

SEQ ID NO: 35 - protein, 6 aa, artificial sequence
  Class I PBM
YKQTSV

SEQ ID NO: 36 - protein, 41 aa, artificial sequence
  GCN4p1-GS4-YKQTSV
RMKQLEDKVEELLSKNYHLENEVARLKKLVGGGGSYKQTSV

SEQ ID NO: 37 - protein, 41 aa, artificial sequence
  GCN4p1(7P14P)-GS4-YKQTSV
RMKQLEPKVEELLPKNYHLENEVARLKKLVGGGGSYKQTSV

SEQ ID NO: 38 - protein, 40 aa, artificial sequence
  GCN4-GS4-GS4
RMKQLEDKVEELLSKNYHLENEVARLKKLVGGGGSGGGGS

SEQ ID NO: 39 - protein, 40 aa, artificial sequence
  GCN4-GS4-V-to-D
RMKQLEDKVEELLSKNYHLENEVARLKKLVGGGGSHWLKD

SEQ ID NO: 40 - protein, 5 aa, artificial sequence
  Class III PBM
KVDSV

SEQ ID NO: 41 - protein, 5 aa, artificial sequence
  Class III PBM
GKDYV

SEQ ID NO: 42 - protein, 5 aa, artificial sequence
  Class III PBM
RKDYV

SEQ ID NO: 43 - protein, 5 aa, artificial sequence
  Class III PBM
TAEMF

SEQ ID NO: 44 - protein, 5 aa, artificial sequence
  Class III PBM
QEDGA

SEQ ID NO: 45 - protein, 33 aa, artificial sequence
  (alpha4 helix of the SSO10a protein
GEELLEDIRKFNEMRKNMDQLKEKINSVLSIRQ

SEQ ID NO: 46 - protein, 43 aa, artificial sequence
  SSO10a-GS4-HWLKV
GEELLEDIRKFNEMRKNMDQLKEKINSVLSIRQGGGGSHWLKV

SEQ ID NO: 47 - protein, 22 aa, artificial sequence
  example leucine zipper
LVKAEHGLDKVEEQLEVARAKL

SEQ ID NO: 48 - protein, 4 aa, artificial sequence
  gLinker3
GGGS

SEQ ID NO: 49 - protein, 6 aa, artificial sequence
  gLinker5
GGGGSG

SEQ ID NO: 50 - protein, 7 aa, artificial sequence
  gLinker6
GGGGSGG